SEQUENCE LISTING (66 sequences)

SEQ ID NO: 1 - DNA, 23 bp, Artificial
  Mstn on target M3 protospacer and PAM
aagtctctccgggacctcttggg

SEQ ID NO: 2 - DNA, 23 bp, Artificial
  chr16+3906202 protospacer and PAM
aaggctctccaggacctcttggg

SEQ ID NO: 3 - DNA, 23 bp, Artificial
  Locus chr4-55206243 protospacer and PAM
aagtatctctgggacctcttcag

SEQ ID NO: 4 - DNA, 23 bp, Artificial
  Locus chr13+38093999 protospacer and PAM
gagtccctccgggagctcttggg

SEQ ID NO: 5 - DNA, 23 bp, Artificial
  Locus Mstn on target M4 protospacer and PAM
gtgctgccgctaccccctcacgg

SEQ ID NO: 6 - DNA, 23 bp, Artificial
  Locus chr2-49015581 protospacer and PAM
gtgctgcagctgccacctcaggg

SEQ ID NO: 7 - DNA, 23 bp, Artificial
  Locus chr5+132154281 protospacer and PAM
gtgcagccgctaccaccacaaag

SEQ ID NO: 8 - protein, 1368 aa, Streptococcus pyogenes [sequence omitted]

SEQ ID NO: 9 - DNA, 40 bp, Artificial
  gRNA spacer sequence
gggccatgtggacatccatgaggtgagacagtgccagcgt

SEQ ID NO: 10 - DNA, 37 bp, Artificial
  gRNA spacer sequence
ggcctgaagccactacagctgctggagatcaaggctc

SEQ ID NO: 11 - DNA, 38 bp, Artificial
  gRNA spacer sequence
ggcctgaagccactacagctgctggagatcaaggctcg

SEQ ID NO: 12 - DNA, 30 bp, Artificial
  gRNA spacer sequence
gccattgcagctgttagaagtgaaagcaag

SEQ ID NO: 13 - DNA, 27 bp, Artificial
  gRNA spacer sequence
ggccctagcatctaagttctcgcaggc

SEQ ID NO: 14 - DNA, 35 bp, Artificial
  gRNA spacer sequence
ggtcattccatctcagctgtgacagcagcgcagaa

SEQ ID NO: 15 - DNA, 34 bp, Artificial
  gRNA spacer sequence
ggaagtcaaggtgacagacacacccaagaggtcc

SEQ ID NO: 16 - DNA, 31 bp, Artificial
  gRNA spacer sequence
ggacacacccaagaggtcccggagagacttt

SEQ ID NO: 17 - DNA, 30 bp, Artificial
  gRNA spacer sequence
gtcaagcccaaagtctctccgggacctctt

SEQ ID NO: 18 - DNA, 29 bp, Artificial
  gRNA spacer sequence
ggaatcccggtgctgccgctaccccctca

SEQ ID NO: 19 - DNA, 21 bp, Artificial
  gRNA spacer sequence
gctagagaataggaacttctt

SEQ ID NO: 20 - DNA, 21 bp, Artificial
  gRNA spacer sequence
gaaagaattgatttgataccg

SEQ ID NO: 21 - DNA, 19 bp, Artificial
  gRNA spacer sequence
gatccccatcaagctgatc

SEQ ID NO: 22 - DNA, 21 bp, Artificial
  gRNA spacer sequence
ggtatgctatacgaagttatt

SEQ ID NO: 23 - DNA, 402 bp, Artificial
  chimeric gRNA scaffold
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttt

SEQ ID NO: 24 - DNA, 54 bp, Artificial
  Amplification primer
  misc_feature (29)..(34): n is a, c, g, or t
ctttccctacacgacgctcttccgatctnnnnnnctggagtgttagagtgggcg

SEQ ID NO: 25 - DNA, 48 bp, Artificial
  Amplification primer
ggagttcagacgtgtgctcttccgatctgactgccccatggaaagaca

SEQ ID NO: 26 - DNA, 58 bp, Artificial
  Amplification primer
  misc_feature (29)..(34): n is a, c, g, or t
ctttccctacacgacgctcttccgatctnnnnnngggccatgaaaggaaaaatgaagt

SEQ ID NO: 27 - DNA, 48 bp, Artificial
  Amplification primer
ggagttcagacgtgtgctcttccgatctgcctctggggtttgcttggt

SEQ ID NO: 28 - DNA, 67 bp, Artificial
  Amplification primer
  misc_feature (29)..(34): n is a, c, g, or t
ctttccctacacgacgctcttccgatctnnnnnngagatataagctgaataaggccaatgacatact

SEQ ID NO: 29 - DNA, 67 bp, Artificial
  Amplification primer
  misc_feature (29)..(34): n is a, c, g, or t
ctttccctacacgacgctcttccgatctnnnnnnggtatgtttattgaaattccctagtctatctac

SEQ ID NO: 30 - DNA, 48 bp, Artificial
  Amplification primer
ggagttcagacgtgtgctcttccgatctctactgctctttcctgccga

SEQ ID NO: 31 - DNA, 54 bp, Artificial
  Amplification primer
ggagttcagacgtgtgctcttccgatctaaatacagaagtagatagactaggga

SEQ ID NO: 32 - DNA, 130 bp, Artificial
  AAV ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 33 - protein, 831 aa, Artificial
  Coding sequence for SphCas9N-RmaIntN
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 34 - DNA, 2496 bp, Artificial
  Coding sequence for SphCas9N-RmaIntN
atggccccaaagaagaagcggaaggtcggtatccacggagtcccagcagccgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttgtctggctggcgatactctcattaccctggccgatggacgacgagtgcctattagagaactggtgtcacagcagaatttttccgtgtgggctctgaatcctcagacttaccgcctggagagggctagagtgagtagagctttctgtaccggcatcaaacctgtgtaccgcctcaccactagactggggagatccattagggccactgccaaccaccgatttctcacacctcagggctggaaacgagtcgatgaactccagcctggagattacctggctctgcctaggagaatccctactgcctcctga

SEQ ID NO: 35 - protein, 970 aa, Artificial
  Coding sequence for RmaIntC-SphCas9C-P2A- turboGFP
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVSRAGSGATNFSLLKQAGDVEENPGPMPAMKIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAR

SEQ ID NO: 36 - DNA, 2913 bp, Artificial
  Coding sequence for RmaIntC-SphCas9C-P2A- turboGFP
atggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtctcgagctggatccggagccacgaacttctctctgttaaagcaagcaggggacgtggaagaaaaccccggtcctatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 37 - DNA, 21 bp, Artificial
  qPCR primers
ggaacccctagtgatggagtt

SEQ ID NO: 38 - DNA, 16 bp, Artificial
  qPCR primer
cggcctcagtgagcga

SEQ ID NO: 39 - DNA, 21 bp, Artificial
  qPCR probe
cactccctctctgcgcgctcg

SEQ ID NO: 40 - DNA, 54 bp, Artificial; identical to SEQ ID NO: 24
  Primer
  misc_feature (29)..(34): n is a, c, g, or t

SEQ ID NO: 41 - DNA, 48 bp, Artificial; identical to SEQ ID NO: 25
  Primer

SEQ ID NO: 42 - DNA, 58 bp, Artificial; identical to SEQ ID NO: 26
  Primer
  misc_feature (29)..(34): n is a, c, g, or t

SEQ ID NO: 43 - DNA, 48 bp, Artificial; identical to SEQ ID NO: 27
  Primer

SEQ ID NO: 44 - DNA, 67 bp, Artificial; identical to SEQ ID NO: 28
  Primer
  misc_feature (29)..(34): n is a, c, g, or t

SEQ ID NO: 45 - DNA, 48 bp, Artificial; identical to SEQ ID NO: 30
  Primer

SEQ ID NO: 46 - DNA, 40 bp, Artificial; identical to SEQ ID NO: 9
  gRNA spacer sequence

SEQ ID NO: 47 - DNA, 38 bp, Artificial; identical to SEQ ID NO: 11
  gRNA spacer sequence

SEQ ID NO: 48 - DNA, 27 bp, Artificial
  gRNA spacer sequence
ggccctagcatctaagttctcgcaggc

SEQ ID NO: 49 - DNA, 35 bp, Artificial; identical to SEQ ID NO: 14
  gRNA spacer sequence

SEQ ID NO: 50 - DNA, 30 bp, Artificial; identical to SEQ ID NO: 17
  gRNA spacer sequence

SEQ ID NO: 51 - DNA, 29 bp, Artificial
  gRNA spacer sequence
ggaatcccggtgctgccgctaccccctca

SEQ ID NO: 52 - DNA, 21 bp, Artificial
  gRNA spacer sequence
gctagagaataggaacttctt

SEQ ID NO: 53 - DNA, 21 bp, Artificial
  gRNA spacer sequence
gaaagaattgatttgataccg

SEQ ID NO: 54 - DNA, 19 bp, Artificial
  gRNA spacer sequence
gatccccatcaagctgatc

SEQ ID NO: 55 - DNA, 21 bp, Artificial
  gRNA spacer sequence
ggtatgctatacgaagttatt

SEQ ID NO: 56 - DNA, 58 bp, Artificial; identical to SEQ ID NO: 26
  Primer
  misc_feature (29)..(34): n is a, c, g, or t

SEQ ID NO: 57 - DNA, 48 bp, Artificial; identical to SEQ ID NO: 27
  Primer

SEQ ID NO: 58 - DNA, 21 bp, Artificial
  Primer
gcctactcgctgctgcccatt

SEQ ID NO: 59 - DNA, 21 bp, Artificial
  Primer
cctggagacccccaaaagctc

SEQ ID NO: 60 - DNA, 19 bp, Artificial
  Probe
agatcttcccacttcaggt

SEQ ID NO: 61 - DNA, 21 bp, Artificial
  Primer
ggaacccctagtgatggagtt

SEQ ID NO: 62 - DNA, 16 bp, Artificial
  Primer
cggcctcagtgagcga

SEQ ID NO: 63 - DNA, 21 bp, Artificial
  Probe
cactccctctctgcgcgctcg

SEQ ID NO: 64 - DNA, 3181 bp, Artificial
  scAAV-Promoter-Cas9N-RmaIntN-synpA
cagcagctgcgcgctcgctcgctcactgaggccgcccgggcaaagcccgggcgtcgggcgacctttggtcgcccggcctcagtgagcgagcgagcgcgcagagagggagtggggttaaacgttgacattgattattgactagccgctagcaggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattcgccgccaccatggccccaaagaagaagcggaaggtcggtatccacggagtcccagcagccgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttgtctggctggcgatactctcattaccctggccgatggacgacgagtgcctattagagaactggtgtcacagcagaatttttccgtgtgggctctgaatcctcagacttaccgcctggagagggctagagtgagtagagctttctgtaccggcatcaaacctgtgtaccgcctcaccactagactggggagatccattagggccactgccaaccaccgatttctcacacctcagggctggaaacgagtcgatgaactccagcctggagattacctggctctgcctaggagaatccctactgcctcctgacaataaaatatctttattttcattacatctgtgtgttggttttttgtgttagctagcgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccag

SEQ ID NO: 65 - DNA, 2842 bp, Artificial
  scAAV-Promoter- RmaIntC-Cas9C-synpA
cagcagctgcgcgctcgctcgctcactgaggccgcccgggcaaagcccgggcgtcgggcgacctttggtcgcccggcctcagtgagcgagcgagcgcgcagagagggagtggggttaaacgttgacattgattattgactagccgctagcaggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattcgccgccaccatggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgacaataaaatatctttattttcattacatctgtgtgttggttttttgtgttagctagcgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccag

SEQ ID NO: 66 - DNA, 1947 bp, Artificial
  scAAV-Promoter-GFP-SV40pA-U6-gRNA
cagcagctgcgcgctcgctcgctcactgaggccgcccgggcaaagcccgggcgtcgggcgacctttggtcgcccggcctcagtgagcgagcgagcgcgcagagagggagtggggttaaacgttgacattgattattgactagccgctagcaggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccggactctaaggtaaatataaaatttttaagtgtataatgtgttaaactactgattctaattgtttctctcttttagattccaacctttggaactgaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagttgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttttagagctagaaatagcaagttaaaataaggctagtccgttatcaacttgaaaaagtggcaccgagtcggtgcttttttttagctagcgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccag

(19) United States
(12) Patent Application Publication
(10) Pub. No.: US 2018/0155708 A1
(43) Pub. Date: Jun. 7, 2018

(54) Split Cas9 Proteins

(71) Applicant: President and Fellows of Harvard College, Cambridge, MA (US)

(72) Inventors: George M. Church, Brookline, MA (US); Wei Leong Chew, Boston, MA (US)

(21) Appl. No.: 15/541,859

(22) PCT Filed: Jan. 8, 2016
(86) PCT No.: PCT/US16/12570

§ 371 Date: Jul. 6, 2017

Related U.S. Application Data
(60) Provisional application No. 62/246,321, filed on Oct. 26, 2015.
(60) Provisional application No. 62/101,043, filed on Jan. 8, 2015.

(51) Int. Cl. C12N 15/10; C12N 9/22; C12N 15/86

(52) U.S. Cl. CPC C12N 15/1024; C12N 2310/20; C12N 15/86; C12N 9/22

(57) ABSTRACT

Split Cas9 proteins, including an active nuclease, a nickase, and a nuclease-null Cas9 protein, are provided. The Cas9 proteins were derived from nucleic acids encoding the S. pyogenes Cas9 protein. The split Cas9 proteins are provided as a first portion comprising the N-terminal lobe and a second portion comprising the C-terminal lobe. Expression of the split Cas9 proteins utilizes a split intein expression and splicing system derived from Rhodothermus marinus Methods of utilizing the split Cas9 proteins and nucleic acids encoding them for genomic engineering are presented.

### RELATED APPLICATIONS

This application claims priority from U.S. provisional patent application Nos. 62/246,321, filed Oct. 26, 2015, and 62/101,043, filed Jan. 8, 2015, each of which is hereby incorporated herein by reference in its entirety for all purposes.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. elife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR Cas9 is the most well-characterized and widely used. The Cas9-gRNA complex first probes DNA for the protospacer-adjacent motif (PAM) sequence (−NGG for Sp Cas9, or 5′-NRG with a cut site of 2-4 bp 5′ of the PAM, although predominantly 3 bp 5′ of the PAM) after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a blunt double-strand break (DSB) that is predominantly repaired by the non-homologous end joining (NHEJ) pathway or, to a lesser extent, template-directed homologous recombination (HR). It is through the DNA repair following induction of DSBs that enables generation of genetic modifications at defined genomic loci.

### SUMMARY

Aspects of the present disclosure are directed to the use of split Cas9 to perform CRISPR-based methods in cells. According to one aspect, two or more portions or segments of a Cas9 are provided to a cell, such as by being expressed from corresponding nucleic acids introduced into the cell. The two or more portions are combined within the cell to form the Cas9 which has an ability to colocalize with guide RNA at a target nucleic acid. It is to be understood that the Cas9 may have one or more modifications from a full length Cas9 known to those of skill in the art, yet still retain or have the capability of colocalizing with guide RNA at a target nucleic acid. Accordingly, the two or more portions or segments, when joined together, need only produce or result in a Cas9 which has an ability to colocalize with guide RNA at a target nucleic acid.

According to certain general aspects, when a foreign nucleic acid sequence or sequences are expressed by the cell, the two or more portions or segments of an RNA guided DNA binding protein, such as Cas9, are produced and joined together to produce the RNA guided DNA binding protein, such as Cas9. When a foreign nucleic acid sequence or sequences are expressed by the cell, one or more or a plurality of guide RNAs are produced. The RNA guided DNA binding protein, such as Cas9, and a guide RNA produces a complex of the RNA guided DNA binding protein, the guide RNA and a double stranded DNA target sequence. In this aspect, the RNA is said to guide the DNA binding protein to the double stranded DNA target sequence for binding thereto. This aspect of the present disclosure may be referred to as co-localization of the RNA and DNA binding protein to or with the double stranded DNA.

DNA binding proteins within the scope of the present disclosure may include those which create a double stranded break (which may be referred to as a DNA binding protein nuclease), those which create a single stranded break (referred to as a DNA binding protein nickase) or those which have no nuclease activity (referred to as a nuclease null DNA binding protein) but otherwise bind to target DNA. In this manner, a DNA binding protein-guide RNA complex may be used to create a double stranded break at a target DNA site, to create a single stranded break at a target DNA site or to localize a transcriptional regulator or function-conferring protein or domain, which may be expressed by the cell, at a target DNA site so as to regulate expression of target DNA. According to certain aspects, the foreign nucleic acid sequence may encode one or more of a DNA binding protein nuclease, a DNA binding protein nickase or a nuclease null DNA binding protein. The foreign nucleic acid sequence may also encode one or more transcriptional regulator or function-conferring proteins or domains or one or more donor nucleic acid sequences that are intended to be inserted into the genomic DNA. According to one aspect, the foreign nucleic acid sequence encoding an RNA guided nuclease-null DNA binding protein further encodes the transcriptional regulator or function-conferring protein or domain fused to the RNA guided nuclease-null DNA binding protein. According to one aspect, the foreign nucleic acid sequence encoding one or more RNAs further encodes a target of an RNA-binding domain and the foreign nucleic acid encoding the transcriptional regulator or function-conferring protein or domain further encodes an RNA-binding domain fused to the transcriptional regulator or function-conferring protein or domain.

Accordingly, expression of a foreign nucleic acid sequence by a germline cell may result in a double stranded break, a single stranded break and/or transcriptional activation or repression of the genomic DNA. Donor DNA may be inserted at the break site by cell mechanisms such as homologous recombination or nonhomologous end joining. It is to be understood that expression of a foreign nucleic acid sequence as described herein may result in a plurality of double stranded breaks or single stranded breaks at various locations along target genomic DNA, including one or more or a plurality of gene sequences, as desired.

Aspects of the present disclosure are directed a method of providing a cell with a Cas9 protein including providing to the cell one or more foreign nucleic acids encoding a plurality of separate portions or segments of the Cas9 protein, wherein the cell expresses the one or more foreign nucleic acids to produce the plurality of separate portions or segments of the Cas9 protein, and wherein the plurality of separate portions or segments of the Cas9 protein are joined together to form the Cas9 protein active to colocalize with guide RNA to a target nucleic acid.

According to one aspect, the one or more foreign nucleic acids are delivered to the cell by separate vectors. According to one aspect, the one or more foreign nucleic acids are delivered to the cell by separate plasmids or adeno-associated viruses. According to one aspect, the Cas9 is a Type II CRISPR system Cas9. According to one aspect, the plurality of separate portions or segments are connected or joined together by linker pairs. According to one aspect, the plurality of separate portions or segments are connected or joined together by split-intein pairs. According to one aspect, the cell is a eukaryotic cell or prokaryotic cell. According to one aspect, the cell is a bacteria cell, a yeast cell, a mammalian cell, a plant cell or an animal cell. According to one aspect, the Cas9 protein is an enzymatically active Cas9 protein, a Cas9 protein nickase or a nuclease null Cas9 protein (“Cas9Nuc”).

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is a schematic depicting a first nucleic acid encoding a first portion of a Cas9 protein and a second nucleic acid encoding a second portion of the Cas9. The first portion is expressed as a first protein portion. The second portion is expressed as a second protein portion. The first protein portion and the second protein portion are combined together to form the Cas9 protein. Expressing separate nucleic acid encoding separate portions or protein sequences or polypeptides of a Cas9 protein so that the separate portions or protein sequences or polypeptides can be combined into a Cas9 protein may be referred to herein as “split Cas9.” A portion of a Cas9 may be referred to as a “split Cas9” as distinguished from a Cas9 protein sequence sufficient to colocalize with guide RNA at a target nucleic acid, such as a complete or full-length Cas9 sequence as is known in the art or otherwise modified. Cas9 consists of a bilobed structure with the N- and C-termini of the disordered linker indicated. Cas9 is shown bound to the gRNA (red ribbon) and target DNA (blue ribbon).

FIG. 2 depicts graphs demonstrating that reconstituted Cas9 retains nuclease activity similar to full-length Cas9. Left panels: Cas9 without P2A-turboGFP; Right panels: Cas9 with P2A-GFP. Y-axis: Mutational frequency of split Cas9-nuclease versus full-length Cas9-nuclease. X-axis: Titration of the DNA ratio of Cas9N:Cas9C. A total of 400 ng of plasmids encoding gRNAs, and 400 ng of plasmids encoding the split-Cas9 or Cas9FL were used. Red: deletions; Blue: insertions; Purple: indels. (n=2 for each condition).

FIG. 3 depicts images demonstrating that split-Cas9 induces excision-mediated fluorescence-activation in the Ai9 reporter fibroblast cell line, at efficiencies comparable to full-length Cas9 across four gRNA pairs. Fluorescence activation was not detected in Cas9 only (no gRNA) controls. Fluorescence activation was observed in a small number of cells co-transfected with Cas9 and single-gRNA, or Cas9 with paired-gRNAs both targeting only one side of the LSL cassette. A 1:1 mass ratio of plasmids encoding Cas9^{N}:Cas9^{C} was used. Red: tdTomato. Green: turboGFP. Scale bar: 200 um.

FIG. 4 is a schematic showing a first nucleic acid encoding a first portion of a Cas9 protein and a second nucleic acid encoding a second portion of the Cas9 as AAV-CRISPR contructs. Cassettes are flanked by AAV ITRs (dark gray).

FIG. 5 depicts images demonstrating transduction efficiency, as detected via P2A-turboGFP, and as correlated with the amount of AAV-containing lysate added to the differentiated C2C12 myotubes.

FIG. 6 depicts graphs of data demonstrating that AAV-CRISPR is active against endogenous genes. mMstn, mActRIIB, and mActRIIA were targeted in C2C12 myotubes. A Cas9N:Cas9C ratio of 1:1 was used in all experiments. Genotyping was conducted 7 days post-transduction. Each blue dot represents the mutational frequency detected per replicate per condition.

FIG. 7, FIG. 8 and FIG. 9 depicts data showing that AAV-CRISPR activated the LSL-tdTomato reporter. Application of lysates containing AAV-CRISPR induced excision-dependent fluorescence-activation in the fluorescence-activation reporter cell line (n=5). Images were taken 7 days post-tranduction.

FIG. 10 depicts transduction of C2C12 myotubes with AAV-DJ-CRISPR purified via density-gradient ultracentrifugation. The myostatin gene was targeted by simultaneous expression of gRNAs mMstn3 and mMstn4. Total AAV titers are kept at 10¹², while the ratio of Cas9^{C}-P2A-turboGFP:Cas9^{N}-U6-gRNAs varied as stated.

FIG. 11 depicts that transduction of LSL-tdTomato reporter cell line with AAV-DJ-CRISPR induced excision-mediated fluorescence activation. All images were taken 4 days post-transduction.

FIG. 12 depicts transduction of GC-1 spermatogonial stem cells with AAV serotype 9. Crude lysates containing scAAV-9 encoding CMV-EGFP-U6-Sp gRNAs (mMstn3 and mMstn4) were applied to GC-1 cells at stated volumes, and GFP fluorescence imaged daily. Transduction was observed in all tested volumes, and GFP intensity peaked at 2 days post-transduction, followed by a plateau through day 4. Scale bar=200 um.

FIG. 13 depicts encoding gRNAs in self-complementary AAVs (scAAVs). 50 ul of each AAV-containing lysate was applied to C2C12 myotubes. Negative control consists of 50 ul of lysate from producer 293AAV cells that were transfected with pRepCap-DJ and pAAV, but with pHelper omitted, which is not expected to produce infectious virions. Absence of transduction in negative control indicates that expression of transgene is dependent on infectious AAVs, and not due to uptake of fluorescent proteins in the lysate solution.

FIG. 14 depicts transduction of GC-1 spermatogonial stem cells with scAAV-DJ. scAAV-DJ encoding CMV-EGFP-SV40polyA-U6-gRNA (mMstn3 and mMstn4) was applied at stated titers to GC-1 spg spermatogonial stem cell line. Images were taken 1 day post transduction. Scale bar=200 um.

FIG. 15 is a schematic depicting plasmids encoding split-Cas9. SMVP=promoter; IntN/IntC=split-inteins; NLS=nuclear localization signal; polyA=SV40 polyadenylation signal.

FIG. 16A and FIG. 16B depict the biological activity of split-Cas9 in transfected C2C12 myoblasts. Split-Cas9 was fully active, targeting all endogenous genes tested (Acvr2b, Acvr2a, and Mstn) at efficiencies 85% to 115% of Cas9^{FL}. Split-Cas9 was tested against full-length Cas9 on the three endogenous genes, with or without co-translating P2A-turboGFP, by transfecting C2C12 cells with equal total mass amounts of Cas9 plasmids. Mutation frequencies induced by split-Cas9 and full-length Cas9 are not significantly different across all three genes (one-way ANOVA) (400 ng of total Cas9 plasmids and 400 ng of total gRNAs plasmids). Left panels: Cas9 without P2A-turboGFP (n=3 independent transfections); Right panels: Cas9 with P2A-turboGFP (n=2 independent transfections). Error bars denote the standard error of the mean (s.e.m.).

FIG. 17 depicts images of Split-Cas9 targeting Ai9 fibroblasts equivalently to Cas9^{FL}. Sparse tdTomato+cells were observed with single-gRNA, or paired-gRNAs both targeting one side of 3×Stop (n=2). Td5 and TdL target 5′ of 3×Stop; Td3 and TdR target 3′ of 3×Stop. Gray=tdTomato. Scale bar, 200 μm.

FIG. 18 depicts a schematic of a Cas9 split-site and AAV-CRISPR.

FIG. 19 depicts an expression time course for CRISPR AAV packaged in single-stranded AAV genome (ssAAV). The expression is tightly correlated to Cas9 expression due to the co-translating P2A-turboGFP; onset by 1-2 days post-transduction as detected by co-translating P2A-turboGFP (50 μl of AAV-Cas9C-P2A-turboGFP-containing lysate was applied to the C2C12 myotubes).

FIG. 20A depicts graphs of collected data after 50 μl of AAV-Cas9^{C}-P2A-turboGFP-containing lysate and 50 μl of AAV-Cas9^{N}-U6-gRNAs-containing lysate, or chloroform-ammonium sulfate purified AAV-CRISPR (1E10 vg), were applied to the C2C12 myotubes. Genotyping was conducted 7 days post-transduction. gRNA sequences are the same as those used in transfection by lipofection. Genotyping is conducted as in transfection by lipofection. Each blue dot represents the mutation frequency detected per transduction per condition (P-values, one-tailed Wilcoxon rank-sum against no-gRNA controls, Bonferroni corrected). Red lines denote means±s.e.m.

FIG. 20B depicts an updated version of AAV-CRISPR dose escalation. Iodixanol-purified AAV-CRISPR was applied at varied dosage onto C2C12 myotubes. Genotyping was conducted 7 days post-transduction. AAV-CRISPR transduces and edits cultured myotubes. At each functional Cas9^{N}:Cas9^{C} tested, mutation frequency increased with AAV dose (P<0.001, one-way ANOVA, Holm-Šidák test), but began to plateau at ˜6% (n.s., not significant between 1E11 and 1E12). Error bars denote s.e.m.

FIG. 21 depicts a graph of AAV-CRISPR^{M3+M4} Mstn gene edits in GC-1 spermatogonial cells (Cas9N:Cas9C, 1:1) (*, P<0.05, Welch's t-test, Bonferroni corrected).

FIG. 22 depicts that transduction of Ai9 tail-tip fibroblasts with 1E12 vg of AAV-CRISPR targeting the 3×Stop cassette induced excision-dependent fluorescence activation. All images were taken 7 days post-transduction. Ratio denotes AAV-Cas9^{N}-U6-gRNAs:AAV-Cas9^{C}-P2A-turboGFP applied. Scale bars, 500 μm.

FIG. 23A schematically depicts neonatal mice injected with AAV9-CRISPR targeting Mstn (AAV9-CRISPR^{M3+M4}). FIG. 23B, FIG. 23C, and FIG. 23D graphically depict the results of systematically delivered AAV9-CRISPR to genetically modify organs. The data includes mutation frequency that reflects viral transduction efficiency. FIG. 23B graphically depicts the data from the graphs in FIG. 23C and FIG. 23D. The data from FIG. 23C depicts deep-sequencing of tissues and indicates Mstn gene-targeting rates ranging from 7.8% to 0.25% (n=4 mice injected with 4E12 of AAV9-CRISPR^{M3+M4}) (*, P<0.05, Wilcoxon rank-sum against controls, Bonferroni corrected). Error bars denote s.e.m. FIG. 23C data is plotted on the y-axis of FIG. 23B. FIG. 23D depicts how AAV9 preferentially transduces the liver, heart, and skeletal muscle (gastrocnemius and diaphragm) (***, P<0.001; Wilcoxon rank-sum, Bonferroni corrected). Each dot represents respective tissue from each mouse (n=7 mice injected with 4E12 of AAV9-CRISPR). Red lines=means±s.e.m.; black dashed line with gray box=qPCR false positive rate with s.d. (2.5 vg/diploid). FIG. 23D data is plotted on the x-axis of FIG. 23B. A total of 4E12 AAV9s were injected intraperitoneally per 3-day old neonatal mouse.

FIG. 24 graphically depicts putative off-target sites within the mouse genome. Results are ranked according to the number of mismatches against the on-target sequence, and CRISPR-targeting at those most similar was assessed by deep sequencing (n=4 mice injected with 4E12 of AAV9-CRISPR^{M3+M4}, and n=2 control mice injected with 4E12 of AAV9-CRISPR^{TdL+TdR} for determination of sequencing error rates). Mismatches are highlighted in red. (SEQ ID NO: 1-7).

FIG. 25A graphically depicts transduction efficiency following 5E11 of AAV9-CRISPR injected (**, P<0.01; ***, P<0.001; Wilcoxon rank-sum, Bonferroni corrected) (n=9 mice). FIG. 25B graphically depicts decreases in mutation frequencies in organs as a result of the reduction of injected viral dose (n=2 mice injected with 5E11 of AAV9-CRISPR^{M3+M4}). Error bars denote s.e.m.

FIG. 26 depicts fluorescent images of AAV9-CRISPR^{TdL+TdR}-edited tdTomato+cells detected in multiple organs (2 upper rows) (n=3 mice at 4E12), and absent in mice injected with AAV9-CRISPR^{M3+M4} (2 lower rows) (n=4 mice at 4E12). Gray=tdTomato. Scale bar, 5 mm.

FIG. 27A, FIG. 27B, and FIG. 27C depict images of tissue sections from mice injected with AAV9-CRISPR. AAV9-CRISPR^{TdL+TdR} (4E12 vg) transduces multiple organs, excising the 3×Stop genomic locus, as indicated by tdTomato activation in the liver (FIG. 27A), heart (FIG. 27B), and skeletal muscle (FIG. 27C). TdTomato+cells were not detected in control mice injected with AAV9-CRISPR^{M3+M4}. Scale bars, 500 μm.

FIG. 28 depicts epifluorescent images of the maternal transmission of AAV9-GFP-Cre that results in mosaic genetic modifications in all offspring. Pregnant mice were injected intravenously with AAV9 carrying gene-editing cargoes via the tail-vein (n=2 mice injected per condition), and delivered pups were examined for genetically modified cells. Two pups are shown per mother, and are representative of all littermates. AAV9-GFP-Cre was maternally transmitted to all offspring, resulting in mosaic loxP recombination within the liver, heart, and skeletal muscles of all progeny. At current efficiencies, AAV9-CRISPR^{TdL+TdR} did not result in gene-edited tdTomato+cells within the progeny. Gray=tdTomato.

FIG. 29 depicts reconstituted Cas9FL by Split-Cas9 at 50% efficiency when delivered intramuscularly.

FIG. 30 depicts the presence of viral genomic bands at the expected 3.2 kb and 2.8 kb sizes for scAAV-Cas9N and scAAV-Cas9C indicating that the split-Cas9 components were packaged into the viruses intact.

### DETAILED DESCRIPTION

Embodiments of the present disclosure are directed to a method of providing a cell with a Cas9 protein comprising providing to the cell a first nucleic acid encoding a first portion of the Cas9 protein and a second nucleic acid encoding a second portion of the Cas9 protein, wherein the cell expresses the first nucleic acid encoding the first portion of the Cas9 protein, wherein the cell expresses the second nucleic acid encoding the second portion of the Cas9 protein, wherein the first portion of the Cas9 protein and the second portion of the Cas9 protein are joined together to form the Cas9 protein. According to one aspect, the first nucleic acid and the second nucleic acid are delivered to the cell by separate vectors. According to one aspect, the first nucleic acid is delivered to the cell by a plasmid or adeno-associated virus. According to one aspect, the second nucleic acid is delivered to the cell by a plasmid or an adeno-associated virus. According to one aspect, the Cas9 is a Type II CRISPR system Cas9.

According to one aspect, the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

According to one aspect, the first nucleic acid encodes a first portion of the Cas9 protein having a Rhodothermus marinus N-split-intein RmaIntN and wherein the second nucleic acid encodes a second portion of the Cas9 protein having a Rhodothermus marinus C-split-intein RmaIntC and wherein the first portion of the Cas9 protein and the second portion of the Cas9 protein are joined together to form the Cas9 protein.

According to one aspect, the first portion of the Cas9 protein is the N-terminal lobe of the Cas9 protein and the second portion of the Cas9 protein is the C-terminal lobe of the Cas9 protein.

According to one aspect, the first portion of the Cas9 protein is the N-terminal lobe of the Cas9 protein up to amino acid V713 and the second portion of the Cas9 protein is the C-terminal lobe of the Cas9 protein beginning at D714.

According to one aspect, the cell is a eukaryotic cell or a prokaryotic cell. According to one aspect, the cell is a bacteria cell, a yeast cell, a mammalian cell, a plant cell or an animal cell.

According to one aspect, the Cas9 protein is an enzymatically active Cas9 protein, a Cas9 protein nickase or a nuclease null Cas9 protein.

Embodiments of the present disclosure are directed to a method of altering a target nucleic acid in a eukaryotic cell comprising providing to the cell a first nucleic acid encoding a first portion of a Cas9 protein and a second nucleic acid encoding a second portion of the Cas9 protein, providing to the cell a third nucleic acid encoding RNA complementary to the target nucleic acid, wherein the cell expresses the RNA, the first portion of the Cas9 protein and the second portion of the Cas9 protein, wherein the first portion of the Cas9 protein and the second portion of the Cas9 protein are joined together to form the Cas9 protein, wherein the RNA and the Cas9 protein form a co-localization complex with the target nucleic acid.

According to one aspect, the Cas9 protein is enzymatically active and the enzymatically active Cas9 protein cleaves the target nucleic acid in a site specific manner.

According to one aspect, the first nucleic acid and the second nucleic acid are delivered to the cell by separate vectors.

According to one aspect, the first nucleic acid is delivered to the cell by a plasmid or adeno-associated virus.

According to one aspect, the second nucleic acid is delivered to the cell by a plasmid or an adeno-associated virus.

According to one aspect, the Cas9 is a Type II CRISPR system Cas9.

According to one aspect, the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

According to one aspect, the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein.

According to one aspect, the first portion of the Cas9 protein is the N-terminal lobe of the Cas9 protein and the second portion of the Cas9 protein is the C-terminal lobe of the Cas9 protein.

According to one aspect, the first portion of the Cas9 protein is the N-terminal lobe of the Cas9 protein up to amino acid V713 and the second portion of the Cas9 protein is the C-terminal lobe of the Cas9 protein beginning at D714.

According to another aspect, the split Cas9 can also be from other Cas9 orthologs to include SpCas9, AnCas9, and SaCas9. All three orthologs have a bi-lobed structure. This consistent feature is likely present in other Cas9 orthologs with yet undetermined structures. See Nishimasu, H. et al. Crystal structure of Cas9 in complex with guide RNA and target DNA. Cell 156, 935-949 (2014), Jinek, M. et al. Structures of Cas9 endonucleases reveal RNA-mediated conformational activation. Science 343, 1247997 (2014), Nishimasu, H. et al. Crystal Structure of Staphylococcus aureus Cas9. Cell 162, 1113-1126 (2015).

According to one aspect, the cell is a eukaryotic cell or a prokaryotic cell. According to one aspect, the cell is a bacteria cell, a yeast cell, a mammalian cell, a plant cell or an animal cell.

According to one aspect, the Cas9 protein is an enzymatically active Cas9 protein, a Cas9 protein nickase or a nuclease null Cas9 protein.

Embodiments of the present disclosure are directed to a cell comprising a first foreign nucleic acid encoding a first portion of a Cas9 protein and a second nucleic acid encoding a second portion of the Cas9 protein, and a third foreign nucleic acid encoding one or more RNAs complementary to DNA, wherein the DNA includes a target nucleic acid, wherein the one or more RNAs, the Cas9 protein are members of a co-localization complex for the target nucleic acid.

Embodiments of the present disclosure are directed to a method of delivering a Cas9 protein to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a first nucleic acid encoding a first portion of the Cas9 protein wherein the first nucleic acid is within a first vector and intravenously administering to the subject a second nucleic acid encoding a second portion of the Cas9 protein wherein the second nucleic acid is within a second vector, wherein the first vector delivers the first nucleic acid to a cell and wherein the second vector delivers the second nucleic acid to the cell, wherein the cell expresses the first nucleic acid encoding the first portion of the Cas9 protein, wherein the cell expresses the second nucleic acid encoding the second portion of the Cas9 protein, wherein the first portion of the Cas9 protein and the second portion of the Cas9 protein are joined together to form the Cas9 protein.

According to one aspect, the first vector is a plasmid or adeno-associated virus.

According to one aspect, the second vector is a plasmid or adeno-associated virus.

According to one aspect, the Cas9 is a Type II CRISPR system Cas9.

According to one aspect, the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

According to one aspect, the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein.

According to one aspect, the first portion of the Cas9 protein is the N-terminal lobe of the Cas9 protein and the second portion of the Cas9 protein is the C-terminal lobe of the Cas9 protein.

According to one aspect, the first portion of the Cas9 protein is the N-terminal lobe of the Cas9 protein up to amino acid V713 and the second portion of the Cas9 protein is the C-terminal lobe of the Cas9 protein beginning at D714.

According to one aspect, the cell is a eukaryotic cell. According to one aspect, the cell is a mammalian cell.

According to one aspect, the Cas9 protein is an enzymatically active Cas9 protein, a Cas9 protein nickase or a nuclease null Cas9 protein.

The split Cas9 methods described herein are useful in CRISPR-related methods where Cas9 and a guide RNA are used to colocalize the Cas9 and the guide RNA to a target nucleic acid sequence. Accordingly, embodiments of the present disclosure are based on the use of RNA guided DNA binding proteins, such as Cas9, to co-localize with guide RNA at a target DNA site. Such DNA binding proteins are readily known to those of skill in the art to bind to DNA for various purposes. Such DNA binding proteins may be naturally occurring. DNA binding proteins included within the scope of the present disclosure include those which may be guided by RNA, referred to herein as guide RNA. According to one aspect, the guide RNA is between about 10 to about 500 nucleotides. According to one aspect, the RNA is between about 20 to about 100 nucleotides. According to this aspect, the guide RNA and the RNA guided DNA binding protein form a co-localization complex at the DNA.

DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. Exemplary Cas include S. pyogenes Cas9 (SpCas9), S. aureus Cas9 (SaCas9) and S. thermophilus Cas9 (StCas9).

Bacterial and archaeal CRISPR-Cas systems rely on short guide RNAs in complex with Cas proteins to direct degradation of complementary sequences present within invading foreign nucleic acid¹. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February, 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

Exemplary DNA binding proteins having nuclease activity function to nick or cut double stranded DNA. Such nuclease activity may result from the DNA binding protein having one or more polypeptide sequences exhibiting nuclease activity. Such exemplary DNA binding proteins may have two separate nuclease domains with each domain responsible for cutting or nicking a particular strand of the double stranded DNA. Exemplary polypeptide sequences having nuclease activity known to those of skill in the art include the McrA-HNH nuclease related domain and the RuvC-like nuclease domain. Accordingly, exemplary DNA binding proteins are those that in nature contain one or more of the McrA-HNH nuclease related domain and the RuvC-like nuclease domain.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is shown below. See Deltcheva et al., Nature 471, 602-607 (2011) hereby incorporated by reference in its entirety.

(SEQ ID NO: 8)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

According to one aspect, the specificity of gRNA-directed Cas9 cleavage is used as a mechanism for genome engineering. According to one aspect, hybridization of the gRNA need not be 100 percent in order for the enzyme to recognize the gRNA/DNA hybrid and affect cleavage. Some off-target activity could occur. For example, the S. pyogenes system tolerates mismatches in the first 6 bases out of the 20 bp mature spacer sequence in vitro. According to one aspect, greater stringency may be beneficial in vivo when potential off-target sites matching (last 14 bp) NGG exist within the human reference genome for the gRNAs.

According to certain aspects, specificity may be improved. When interference is sensitive to the melting temperature of the gRNA-DNA hybrid, AT-rich target sequences may have fewer off-target sites. Carefully choosing target sites to avoid pseudo-sites with at least 14 bp matching sequences elsewhere in the genome may improve specificity. The use of a Cas9 variant requiring a longer PAM sequence may reduce the frequency of off-target sites. Directed evolution may improve Cas9 specificity to a level sufficient to completely preclude off-target activity, ideally requiring a perfect 20 bp gRNA match with a minimal PAM. Accordingly, modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

Guide RNAs useful in the disclosed methods include those having a spacer sequence, a tracr mate sequence and a tracr sequence, with the spacer sequence being between about 16 to about 20 nucleotides in length and with the tracr sequence being between about 60 to about 500 nucleotides in length and with a portion of the tracr sequence being hybridized to the tracr mate sequence and with the tracr mate sequence and the tracr sequence being linked by a linker nucleic acid sequence of between about 4 to about 6 nucleotides. crRNA-tracrRNA fusions are contemplated as exemplary guide RNA.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase.

An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null Cas9 protein.

According to certain aspects of methods of RNA-guided genome regulation described herein, Cas9 is altered to reduce, substantially reduce or eliminate nuclease activity. According to one aspect, Cas9 nuclease activity is reduced, substantially reduced or eliminated by altering the RuvC nuclease domain or the HNH nuclease domain. According to one aspect, the RuvC nuclease domain is inactivated. According to one aspect, the HNH nuclease domain is inactivated. According to one aspect, the RuvC nuclease domain and the HNH nuclease domain are inactivated. According to an additional aspect, Cas9 proteins are provided where the RuvC nuclease domain and the HNH nuclease domain are inactivated. According to an additional aspect, nuclease-null Cas9 proteins are provided insofar as the RuvC nuclease domain and the HNH nuclease domain are inactivated. According to an additional aspect, a Cas9 nickase is provided where either the RuvC nuclease domain or the HNH nuclease domain is inactivated, thereby leaving the remaining nuclease domain active for nuclease activity. In this manner, only one strand of the double stranded DNA is cut or nicked.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. pyogenes and protein sequences having at least 30%, 40%, 50%, 60%, 70%, 80%, 90%, 95%, 98% or 99% homology thereto and being a DNA binding protein, such as an RNA guided DNA binding protein.

According to one aspect, an engineered Cas9-gRNA system is provided wherein one or more of function-conferring domains, such as FokI heterodimers (see Tsai, S. Q., et al., Dimeric CRISPR RNA-guided FokI nucleases for highly specific genome editing. Nat Biotechnol, 2014. 32(6): p. 569-76, and Guilinger, J. P., D. B. Thompson, and D. R. Liu, Fusion of catalytically inactive Cas9 to FokI nuclease improves the specificity of genome modification. Nat Biotechnol, 2014. 32(6): p. 577-82.), transcriptional regulators (see Gilbert, L. A., et al., CRISPR-mediated modular RNA-guided regulation of transcription in eukaryotes. Cell, 2013. 154(2): p. 442-51, Mali, P., et al., CAS9 transcriptional activators for target specificity screening and paired nickases for cooperative genome engineering. Nat Biotechnol, 2013. 31(9): p. 833-8, Perez-Pinera, P., et al., RNA-guided gene activation by CRISPR-Cas9-based transcription factors. Nat Methods, 2013. 10(10): p. 973-6, and Cheng, A. W., et al., Multiplexed activation of endogenous genes by CRISPR-on, an RNA-guided transcriptional activator system. Cell Res, 2013. 23(10): p. 1163-71), fluorescent proteins (see Gilbert, L. A., et al., CRISPR-mediated modular RNA-guided regulation of transcription in eukaryotes. Cell, 2013. 154(2): p. 442-51 and Chen, B., et al., Dynamic imaging of genomic loci in living human cells by an optimized CRISPR/Cas system. Cell, 2013. 155(7): p. 1479-91), protein-protein interacting-domains (see Tanenbaum, M. E., et al., A protein-tagging system for signal amplification in gene expression and fluorescence imaging. Cell, 2014. 159(3): p. 635-46), and degradation tags are attached to either the Cas9 protein or the gRNA or both for delivery to a target nucleic acid.

According to one aspect, an engineered Cas9-gRNA system is provided which enables RNA-guided genome regulation in cells by tethering transcriptional activation domains to either a nuclease-null Cas9 or to guide RNAs. According to one aspect of the present disclosure, one or more transcriptional regulatory or function-conferring proteins or domains (such terms are used interchangeably) are joined or otherwise connected to a nuclease-deficient Cas9 or one or more guide RNA (gRNA). The transcriptional regulatory or function-conferring domains correspond to targeted loci. Accordingly, aspects of the present disclosure include methods and materials for localizing transcriptional regulatory or function-conferring domains to targeted loci by fusing, connecting or joining such domains to either Cas9Nuc or to the gRNA. According to certain aspects, methods are provided for regulating endogenous genes using Cas9Nuc, one or more gRNAs and a transcriptional regulatory or function-conferring protein or domain. According to one aspect, an endogenous gene can be any desired gene, referred to herein as a target gene.

According to one aspect, a Cas9Nuc-fusion protein capable of transcriptional activation is provided. According to one aspect, a VP64 activation domain (see Zhang et al., Nature Biotechnology 29, 149-153 (2011) hereby incorporated by reference in its entirety) is joined, fused, connected or otherwise tethered to the C terminus of Cas9Nuc. According to one method, the transcriptional regulatory or function-conferring domain is provided to the site of target genomic DNA by the Cas9N protein. According to one method, a Cas9Nuc fused to a transcriptional regulatory or function-conferring domain is provided within a cell along with one or more guide RNAs. The Cas9Nuc with the transcriptional regulatory or function-conferring domain fused thereto bind at or near target genomic DNA. The one or more guide RNAs bind at or near target genomic DNA. The transcriptional regulatory or function-conferring domain regulates expression of the target gene. According to a specific aspect, a Cas9Nuc-VP64 fusion activated transcription of reporter constructs when combined with gRNAs targeting sequences near the promoter, thereby displaying RNA-guided transcriptional activation.

According to one aspect, a gRNA-fusion protein capable of transcriptional activation is provided. According to one aspect, a VP64 activation domain is joined, fused, connected or otherwise tethered to the gRNA. According to one method, the transcriptional regulatory or function-conferring domain is provided to the site of target genomic DNA by the gRNA. According to one method, a gRNA fused to a transcriptional regulatory or function-conferring domain is provided within a cell along with a Cas9Nuc protein. The Cas9Nuc binds at or near target genomic DNA. The one or more guide RNAs with the transcriptional regulatory or function-conferring protein or domain fused thereto bind at or near target genomic DNA. The transcriptional regulatory or function-conferring domain regulates expression of the target gene. According to a specific aspect, a Cas9Nuc protein and a gRNA fused with a transcriptional regulatory or function-conferring domain activated transcription of reporter constructs, thereby displaying RNA-guided transcriptional activation.

According to one aspect, the transcriptional regulator protein or domain is a transcriptional activator. According to one aspect, the transcriptional regulator protein or domain upregulates expression of the target nucleic acid. According to one aspect, the transcriptional regulator protein or domain is a transcriptional repressor. According to one aspect, the transcriptional regulator protein or domain downregulates expression of the target nucleic acid. Transcriptional activators and transcriptional repressors can be readily identified by one of skill in the art based on the present disclosure.

According to one aspect, two or more guide RNAs are provided with each guide RNA being complementary to an adjacent site in the DNA target nucleic acid. At least one RNA guided DNA binding protein nickase is provided and being guided by the two or more RNAs, wherein the at least one RNA guided DNA binding protein nickase co-localizes with the two or more RNAs to the DNA target nucleic acid and nicks the DNA target nucleic acid resulting in two or more adjacent nicks. According to certain aspects, the two or more adjacent nicks are on the same strand of the double stranded DNA. According to one aspect, the two or more adjacent nicks are on the same strand of the double stranded DNA and result in homologous recombination. According to one aspect, the two or more adjacent nicks are on different strands of the double stranded DNA. According to one aspect, the two or more adjacent nicks are on different strands of the double stranded DNA and create double stranded breaks. According to one aspect, the two or more adjacent nicks are on different strands of the double stranded DNA and create double stranded breaks resulting in nonhomologous end joining. According to one aspect, the two or more adjacent nicks are on different strands of the double stranded DNA and are offset with respect to one another. According to one aspect, the two or more adjacent nicks are on different strands of the double stranded DNA and are offset with respect to one another and create double stranded breaks. According to one aspect, the two or more adjacent nicks are on different strands of the double stranded DNA and are offset with respect to one another and create double stranded breaks resulting in nonhomologous end joining. According to one aspect, the two or more adjacent nicks are on different strands of the double stranded DNA and create double stranded breaks resulting in fragmentation of the target nucleic acid thereby preventing expression of the target nucleic acid.

According to certain aspects, binding specificity of the RNA guided DNA binding protein may be increased according to methods described herein. According to one aspect, off-set nicks are used in methods of genome-editing. A large majority of nicks seldom result in NHEJ events, (see Certo et al., Nature Methods 8, 671-676 (2011) hereby incorporated by reference in its entirety) thus minimizing the effects of off-target nicking. In contrast, inducing off-set nicks to generate double stranded breaks (DSBs) is highly effective at inducing gene disruption. According to certain aspects, 5′ overhangs generate more significant NHEJ events as opposed to 3′ overhangs. Similarly, 3′ overhangs favor HR over NHEJ events, although the total number of HR events is significantly lower than when a 5′ overhang is generated. Accordingly, methods are provided for using nicks for homologous recombination and off-set nicks for generating double stranded breaks to minimize the effects of off-target Cas9-gRNA activity.

Target nucleic acids include any nucleic acid sequence to which a co-localization complex as described herein can be useful to either cut, nick or regulate. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA.

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Transcriptional regulator proteins or domains which are transcriptional activators or transcriptional repressors may be readily identifiable by those skilled in the art based on the present disclosure.

Vectors used to deliver the nucleic acids to cells as described herein include vectors known to those of skill in the art and used for such purposes. Certain exemplary vectors may be plasmids or adeno-associated viruses known to those of skill in the art. AAVs are highly prevalent within the human population (see Gao, G., et al., Clades of Adeno-associated viruses are widely disseminated in human tissues. J Virol, 2004. 78(12): p. 6381-8, and Boutin, S., et al., Prevalence of serum IgG and neutralizing factors against adeno-associated virus (AAV) types 1, 2, 5, 6, 8, and 9 in the healthy population: implications for gene therapy using AAV vectors. Hum Gene Ther, 2010. 21(6): p. 704-12) and are useful as viral vectors. Many serotypes exist, each with different tropism for tissue types (see Zincarelli, C., et al., Analysis of AAV serotypes 1-9 mediated gene expression and tropism in mice after systemic injection. Mol Ther, 2008. 16(6): p. 1073-80), which allows specific tissues to be preferentially targeted with appropriate pseudotyping. Some serotypes, such as serotypes 8, 9, and rh10, transduce the mammalian body. See Zincarelli, C., et al., Analysis of AAV serotypes 1-9 mediated gene expression and tropism in mice after systemic injection. Mol Ther, 2008. 16(6): p. 1073-80, Inagaki, K., et al., Robust systemic transduction with AAV9 vectors in mice: efficient global cardiac gene transfer superior to that of AAV8. Mol Ther, 2006. 14(1): p. 45-53, Keeler, A. M., et al., Long-term correction of very long-chain acyl-coA dehydrogenase deficiency in mice using AAV9 gene therapy. Mol Ther, 2012. 20(6): p. 1131-8, Gray, S. J., et al., Preclinical differences of intravascular AAV9 delivery to neurons and glia: a comparative study of adult mice and nonhuman primates. Mol Ther, 2011. 19(6): p. 1058-69, Okada, H., et al., Robust Long-term Transduction of Common Marmoset Neuromuscular Tissue With rAAV1 and rAAV9. Mol Ther Nucleic Acids, 2013. 2: p. e95, and Foust, K. D., et al., Intravascular AAV9 preferentially targets neonatal neurons and adult astrocytes. Nat Biotechnol, 2009. 27(1): p. 59-65. AAV9 has been demonstrated to cross the blood-brain barrier (see Foust, K. D., et al., Intravascular AAV9 preferentially targets neonatal neurons and adult astrocytes. Nat Biotechnol, 2009. 27(1): p. 59-65, and Rahim, A. A., et al., Intravenous administration of AAV2/9 to the fetal and neonatal mouse leads to differential targeting of CNS cell types and extensive transduction of the nervous system. FASEB J, 2011. 25(10): p. 3505-18) that is inaccessible to many viral vectors and biologics. Certain AAVs have a payload of 4.7-5.0 kb (including viral inverted terminal repeats (ITRs), which are required in cis for viral packaging). See Wu, Z., H. Yang, and P. Colosi, Effect of genome size on AAV vector packaging. Mol Ther, 2010. 18(1): p. 80-6 and Dong, J. Y., P. D. Fan, and R. A. Frizzell, Quantitative analysis of the packaging capacity of recombinant adeno-associated virus. Hum Gene Ther, 1996. 7(17): p. 2101-12.

Delivery methods commonly used in research, such as lentiviruses, adenoviruses, or nucleic-acid-complexes, exhibit substantial immunogenic and cytotoxic properties, which can further compound the immunogenicity from ectopic transgene-expression. Furthermore, these approaches generally lack the capacity for targeting of specific tissues and for robust full-body delivery. To simultaneously minimize pathological impacts and enable systemic genome editing, CRISPR was delivered via adeno-associated viruses (AAVs). AAVs are prevalent within human populations (see Gao, G., et al., Clades of Adeno-associated viruses are widely disseminated in human tissues. J Virol, 2004. 78(12): p. 6381-8), and there have been no established cases of pathology associated with AAV infection, making them one of the most promising vectors currently used in clinical trials. Moreover, tissue-targeting is easily accomplished by pseudotyping to AAV serotypes with suitable tropism. Of particular interest is AAV serotype 9, which robustly transduces multiple cell types in the body (see Zincarelli, C., et al., Analysis of AAV serotypes 1-9 mediated gene expression and tropism in mice after systemic injection. Mol Ther, 2008. 16(6): p. 1073-80. and Foust, K. D., et al., Intravascular AAV9 preferentially targets neonatal neurons and adult astrocytes. Nat Biotechnol, 2009. 27(1): p. 59-65.) and crosses endothelial barriers (e.g. blood-brain barrier, see Foust, K. D., et al., Intravascular AAV9 preferentially targets neonatal neurons and adult astrocytes. Nat Biotechnol, 2009. 27(1): p. 59-65 and Zhang, H. et al. Several rAAV vectors efficiently cross the blood-brain barrier and transduce neurons and astrocytes in the neonatal mouse central nervous system. Mol Ther, 2011 19(8): p. 1440-1448) that block access by other delivery vectors. Together, AAV and CRISPR present an enticing combination for achieving systemic gene-editing, but a key obstacle has been the limited capacity of AAV for packaging exogenous sequences (<4.7 kb). Dong, J. Y., P. D. Fan, and R. A. Frizzell, Quantitative analysis of the packaging capacity of recombinant adeno-associated virus. Hum Gene Ther, 1996. 7(17): p. 2101-12. Of the various Cas9 orthologs that have been co-opted for genome engineering (ST1, Nm, Sa) (see Ran, F. A. et al. In vivo genome editing using Staphylococcus aureus Cas9. Nature, 2015. 520: p. 186-91 and Esvelt, K. M., et al., Orthogonal Cas9 proteins for RNA-guided gene regulation and editing. Nat Methods, 2013. 10(11): p. 1116-21), Sp Cas9 has a least restrictive PAM and most consistent efficacy, but its size (4.2 kb) makes packaging into AAV challenging, necessitating use of a limited repertoire of compact regulatory elements (<500 bp) (see Swiech, L., et al., In vivo interrogation of gene function in the mammalian brain using CRISPR-Cas9. Nat Biotechnol, 2014 and Senis, E. et al. CRISPR/Cas9-mediated genome engineering: an adeno-associated viral (AAV) vector toolbox. Biotechnology journal, 2014, 9: p. 1402-1412), and precluding the fusion of function-conferring domains. The Cas9 protein was re-engineered to eliminate this obstacle.

According to certain aspects, two or more portions of a Cas9 protein are provided within a cell or are otherwise expressed within a cell and are combined together to form the Cas9 protein. This structure-guided design is essential since splitting Cas9 at ordered protein regions significantly impacts function. See Nishimasu, H. et al. Crystal structure of Cas9 in complex with guide RNA and target DNA. Cell 156, 935-949 (2014), Jinek, M. et al. Structures of Cas9 endonucleases reveal RNA-mediated conformational activation. Science 343, 1247997 (2014), Zetsche, B., Volz, S. E. & Zhang, F. A split-Cas9 architecture for inducible genome editing and transcription modulation. Nature biotechnology 33, 139-142 (2015), Nihongaki, Y., Kawano, F., Nakajima, T. & Sato, M. Photoactivatable CRISPR-Cas9 for optogenetic genome editing. Nature biotechnology 33, 755-760 (2015), Nishimasu, H. et al. Crystal Structure of Staphylococcus aureus Cas9. Cell 162, 1113-1126 (2015), and Fine, E. J. et al. Trans-spliced Cas9 allows cleavage of HBB and CCR5 genes in human cells using compact expression cassettes. Scientific reports 5, 10777 (2015).

According to one aspect, two portions of a Cas9 protein are provided within a cell or are otherwise expressed within a cell and are combined together to form the Cas9 protein. The two portions of the Cas9 protein are sufficient in length such that when they are combined into the Cas9 protein, the Cas9 protein has the function of co-localizing at a target nucleic acid with a guide RNA as described above. According to certain aspects, various methods known to those of skill in the art may be used to combine the two or more portions of a Cas9 protein together. Exemplary methods and linkers include split-intein protein trans-splicing for reconstituting the Cas9 protein as is known in the art and as described herein. Other methods include protein-protein interacting domains (see Zakeri, B., et al., Peptide tag forming a rapid covalent bond to a protein, through engineering a bacterial adhesion. Proc Natl Acad Sci USA, 2012. 109(12): p. E690-7 and Fierer, J. O., G. Veggiani, and M. Howarth, SpyLigase peptide-peptide ligation polymerizes affibodies to enhance magnetic cancer cell capture. Proc Natl Acad Sci USA, 2014. 111(13): p. E1176-81) or small molecule dependent interactions. See Los, G. V., et al., HaloTag: a novel protein labeling technology for cell imaging and protein analysis. ACS Chem Biol, 2008. 3(6): p. 373-82 and Keppler, A., et al., A general method for the covalent labeling of fusion proteins with small molecules in vivo. Nat Biotechnol, 2003. 21(1): p. 86-9.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Constructs and Sequences

U6-driven gRNA plasmids were constructed as previously described. See Mali, P., et al., RNA-guided human genome engineering via Cas9. Science, 2013. 339(6121): p. 823-6. AAV plasmid backbone was derived from pZac2.1 (Penn Vector Core), while scAAV plasmid backbone from Addgene #32396 and Addgene #21894. Transgene cassettes were assembled by PCR, IDT gBlocks, and splicing-by-overlap-extension, and inserts were cloned into vector backbones by sticky-end ligation, blunt-end ligation, or Gibson Isothermal Assembly. See Gibson, D. G., et al., Enzymatic assembly of DNA molecules up to several hundred kilobases. Nat Methods, 2009. 6(5): p. 343-5. Minicircles parental plasmids were cloned in ZYCY10P3S2T, and minicircles were generated as in. See Kay, M. A., C. Y. He, and Z. Y. Chen, A robust system for production of minicircle DNA vectors. Nat Biotechnol, 2010. 28(12): p. 1287-9. AAV plasmids were cloned with Stbl3 (Life Technologies). All other plasmids were transformed into DH5a (NEB). All plasmids were verified with Sanger sequencing. Protein transgenes were expressed from ubiquitous hybrid promoters: SMVP promoter (generated by fusing SV40enhancer-CMV-promoter-chimeric intron), CASI promoter (see Balazs, A. B., et al., Antibody-based protection against HIV infection by vectored immunoprophylaxis. Nature, 2012. 481(7379): p. 81-4), or CAG promoter. See Matsuda, T. and C. L. Cepko, Electroporation and RNA interference in the rodent retina in vivo and in vitro. Proc Natl Acad Sci USA, 2004. 101(1): p. 16-22. SMVP promoter was derived from pMAXGFP (Lonza).

gRNA Spacer Sequences:

| | Spacer sequence, including 5′ G |
|---|---|
| Sp gRNAs | from U6 promoter |
| 1st mActRIIB | GGGCCATGTGGACATCCATGAGGTGAGACAGTGC |
| | CAGCGT (SEQ ID NO: 9) |
| 2nd mActRIIB | GGCCTGAAGCCACTACAGCTGCTGGAGATCAAGG |
| | CTC (SEQ ID NO: 10) |
| 3rd mActRIIB | GGCCTGAAGCCACTACAGCTGCTGGAGATCAAGG |
| | CTCG (SEQ ID NO: 11) |
| 1st mActRIIA | GCCATTGCAGCTGTTAGAAGTGAAAGCAAG |
| | (SEQ ID NO: 12) |
| 3rd mActRIIA | GGCCCTAGCATCTAAGTTCTCGCAGGC |
| | (SEQ ID NO: 13) |
| 4th mActRIIA | GGTCATTCCATCTCAGCTGTGACAGCAGCGCAGA |
| | A (SEQ ID NO: 14) |
| 1st mMstn | GGAAGTCAAGGTGACAGACACACCCAAGAGGTCC |
| | (SEQ ID NO: 15) |
| 2nd mMstn | GGACACACCCAAGAGGTCCCGGAGAGACTTT |
| | (SEQ ID NO: 16) |
| 3rd mMstn | GTCAAGCCCAAAGTCTCTCCGGGACCTCTT |
| | (SEQ ID NO: 17) |
| 4th mMstn | GGAATCCCGGTGCTGCCGCTACCCCCTCA |
| | (SEQ ID NO: 18) |
| Ai9 Td5 | GCTAGAGAATAGGAACTTCTT |
| | (SEQ ID NO: 19) |
| Ai9 TdL | GAAAGAATTGATTTGATACCG |
| | (SEQ ID NO: 20) |
| Ai9 Td3 | GATCCCCATCAAGCTGATC |
| | (SEQ ID NO: 21) |
| Ai9 TdR | GGTATGCTATACGAAGTTATT |
| | (SEQ ID NO: 22) |

All Sp. gRNAs were expressed under the U6 promoter, and utilized the chimeric gRNA scaffold (underlined) (see Mali, P., et al., RNA-guided human genome engineering via Cas9. Science, 2013. 339(6121): p. 823-6):

(SEQ ID NO: 23)
TGTACAAAAAAGCAGGCTTTAAAGGAACCAATTCAGTCGACTGGATCCGG
TACCAAGGTCGGGCAGGAAGAGGGCCTATTTCCCATGATTCCTTCATATT
TGCATATACGATACAAGGCTGTTAGAGAGATAATTAGAATTAATTTGACT
GTAAACACAAAGATATTAGTACAAAATACGTGACGTAGAAAGTAATAATT
TCTTGGGTAGTTTGCAGTTTTAAAATTATGTTTTAAAATGGACTATCATA
TGCTTACCGTAACTTGAAAGTATTTCGATTTCTTGGCTTTATATATCTTG
TGGAAAGGACGAAACACCG[spacer]GTTTTAGAGCTAGAAATAGCAAG
TTAAAATAAGGCTAGTCCGTTATCAACTTGAAAAAGTGGCACCGAGTCGG
TGCTTTTTTT

### Locus-Specific Amplification Primers for Deep Sequencing:

| | |
|---|---|
| Primer | Target |

| index | Sequence | locus |
|---|---|---|
| 812 | CTTTCCCTACACGACGCTCTTCCGATCTNNNNNNCTGGAGTGTTAGA | mActRII |
| | GTGGGCG (SEQ ID NO: 24) | B F |
| 813 | GGAGTTCAGACGTGTGCTCTTCCGATCTGACTGCCCCATGGAAAGAC | mActRII |
| | A (SEQ ID NO: 25) | B R |
| 814 | CTTTCCCTACACGACGCTCTTCCGATCTNNNNNNGGGCCATGAAAGG | mMstn F |
| | AAAAATGAAGT (SEQ ID NO: 26) | |
| 815 | GGAGTTCAGACGTGTGCTCTTCCGATCTGCCTCTGGGGTTTGCTTGG | mMstn R |
| | T (SEQ ID NO: 27) | |
| 835 | CTTTCCCTACACGACGCTCTTCCGATCTNNNNNNGAGATATAAGCTG | mActRII |
| | AATAAGGCCAATGACATACT(SEQ ID NO: 28) | A F |
| 837 | CTTTCCCTACACGACGCTCTTCCGATCTNNNNNNGGTATGTTTATTG | mActRII |
| | AAATTCCCTAGTCTATCTAC(SEQ ID NO: 29) | A F |
| 838 | GGAGTTCAGACGTGTGCTCTTCCGATCTCTACTGCTCTTTCCTGCCGA | mActRII |
| | (SEQ ID NO: 30) | A R |
| 751 | GGAGTTCAGACGTGTGCTCTTCCGATCTAAATACAGAAGTAGATAG | mActRII |
| | ACTAGGGA (SEQ ID NO: 31) | A R |

### AAV ITR Sequence:

(SEQ ID NO: 32)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT
Coding sequence for SphCas9^{N}-RmaIntN:
(SEQ ID NO: 33)
MAPKKKRKVGIHGVPAADKKYSIGLDIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVCLAGDTLITLADGRRVPIREL
VSQQNFSVWALNPQTYRLERARVSRAFCTGIKPVYRLTTRLGRSIRATAN
HRFLTPQGWKRVDELQPGDYLALPRRIPTAS.
(SEQ ID NO: 34)
ATGGCCCCAAAGAAGAAGCGGAAGGTCGGTATCCACGGAGTCCCAGCAGC
CGACAAGAAGTACTCCATTGGGCTCGATATCGGCACAAACAGCGTCGGCT
GGGCCGTCATTACGGACGAGTACAAGGTGCCGAGCAAAAAATTCAAAGTT
CTGGGCAATACCGATCGCCACAGCATAAAGAAGAACCTCATTGGCGCCCT
CCTGTTCGACTCCGGGGAAACGGCCGAAGCCACGCGGCTCAAAAGAACAG
CACGGCGCAGATATACCCGCAGAAAGAATCGGATCTGCTACCTGCAGGAG
ATCTTTAGTAATGAGATGGCTAAGGTGGATGACTCTTTCTTCCATAGGCT
GGAGGAGTCCTTTTTGGTGGAGGAGGATAAAAAGCACGAGCGCCACCCAA
TCTTTGGCAATATCGTGGACGAGGTGGCGTACCATGAAAAGTACCCAACC
ATATATCATCTGAGGAAGAAGCTTGTAGACAGTACTGATAAGGCTGACTT
GCGGTTGATCTATCTCGCGCTGGCGCATATGATCAAATTTCGGGGACACT
TCCTCATCGAGGGGGACCTGAACCCAGACAACAGCGATGTCGACAAACTC
TTTATCCAACTGGTTCAGACTTACAATCAGCTTTTCGAAGAGAACCCGAT
CAACGCATCCGGAGTTGACGCCAAAGCAATCCTGAGCGCTAGGCTGTCCA
AATCCCGGCGGCTCGAAAACCTCATCGCACAGCTCCCTGGGGAGAAGAAG
AACGGCCTGTTTGGTAATCTTATCGCCCTGTCACTCGGGCTGACCCCCAA
CTTTAAATCTAACTTCGACCTGGCCGAAGATGCCAAGCTTCAACTGAGCA
AAGACACCTACGATGATGATCTCGACAATCTGCTGGCCCAGATCGGCGAC
CAGTACGCAGACCTTTTTTTGGCGGCAAAGAACCTGTCAGACGCCATTCT
GCTGAGTGATATTCTGCGAGTGAACACGGAGATCACCAAAGCTCCGCTGA
GCGCTAGTATGATCAAGCGCTATGATGAGCACCACCAAGACTTGACTTTG
CTGAAGGCCCTTGTCAGACAGCAACTGCCTGAGAAGTACAAGGAAATTTT
CTTCGATCAGTCTAAAAATGGCTACGCCGGATACATTGACGGCGGAGCAA
GCCAGGAGGAATTTTACAAATTTATTAAGCCCATCTTGGAAAAAATGGAC
GGCACCGAGGAGCTGCTGGTAAAGCTTAACAGAGAAGATCTGTTGCGCAA
ACAGCGCACTTTCGACAATGGAAGCATCCCCCACCAGATTCACCTGGGCG
AACTGCACGCTATCCTCAGGCGGCAAGAGGATTTCTACCCCTTTTTGAAA
GATAACAGGGAAAAGATTGAGAAAATCCTCACATTTCGGATACCCTACTA
TGTAGGCCCCCTCGCCCGGGGAAATTCCAGATTCGCGTGGATGACTCGCA
AATCAGAAGAGACCATCACTCCCTGGAACTTCGAGGAAGTCGTGGATAAG
GGGGCCTCTGCCCAGTCCTTCATCGAAAGGATGACTAACTTTGATAAAAA
TCTGCCTAACGAAAAGGTGCTTCCTAAACACTCTCTGCTGTACGAGTACT
TCACAGTTTATAACGAGCTCACCAAGGTCAAATACGTCACAGAAGGGATG
AGAAAGCCAGCATTCCTGTCTGGAGAGCAGAAGAAAGCTATCGTGGACCT
CCTCTTCAAGACGAACCGGAAAGTTACCGTGAAACAGCTCAAAGAAGACT
ATTTCAAAAAGATTGAATGTTTCGACTCTGTTGAAATCAGCGGAGTGGAG
GATCGCTTCAACGCATCCCTGGGAACGTATCACGATCTCCTGAAAATCAT
TAAAGACAAGGACTTCCTGGACAATGAGGAGAACGAGGACATTCTTGAGG
ACATTGTCCTCACCCTTACGTTGTTTGAAGATAGGGAGATGATTGAAGAA
CGCTTGAAAACTTACGCTCATCTCTTCGACGACAAAGTCATGAAACAGCT
CAAGAGGCGCCGATATACAGGATGGGGGCGGCTGTCAAGAAAACTGATCA
ATGGGATCCGAGACAAGCAGAGTGGAAAGACAATCCTGGATTTTCTTAAG
TCCGATGGATTTGCCAACCGGAACTTCATGCAGTTGATCCATGATGACTC
TCTCACCTTTAAGGAGGACATCCAGAAAGCACAAGTTTGTCTGGCTGGCG
ATACTCTCATTACCCTGGCCGATGGACGACGAGTGCCTATTAGAGAACTG
GTGTCACAGCAGAATTTTTCCGTGTGGGCTCTGAATCCTCAGACTTACCG
CCTGGAGAGGGCTAGAGTGAGTAGAGCTTTCTGTACCGGCATCAAACCTG
TGTACCGCCTCACCACTAGACTGGGGAGATCCATTAGGGCCACTGCCAAC
CACCGATTTCTCACACCTCAGGGCTGGAAACGAGTCGATGAACTCCAGCC
TGGAGATTACCTGGCTCTGCCTAGGAGAATCCCTACTGCCTCCTGA
Coding sequence for RmaIntC-SphCas9^{C}-P2A-turboGFP:
(SEQ ID NO: 35)
MAAACPELRQLAQSDVYWDPIVSIEPDGVEEVFDLTVPGPHNFVANDIIA
HNSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDSRADPKKKRKVSRAGSGATNFSLLKQAGDVEENPGPMPAMKIE
CRITGTLNGVEFELVGGGEGTPEQGRMTNKMKSTKGALTFSPYLLSHVMG
YGFYHFGTYPSGYENPFLHAINNGGYTNTRIEKYEDGGVLHVSFSYRYEA
GRVIGDFKVVGTGFPEDSVIFTDKIIRSNATVEHLHPMGDNVLVGSFART
FSLRDGGYYSFVVDSHMHFKSAIHPSILQNGGPMFAFRRVEELHSNTELG
IVEYQHAFKTPIAFARSRAR.
(SEQ ID NO: 36)
ATGGCGGCGGCGTGCCCGGAACTGCGTCAGCTGGCGCAGAGCGATGTGTA
TTGGGATCCGATTGTGAGCATTGAACCGGATGGCGTGGAAGAAGTGTTTG
ATCTGACCGTGCCGGGCCCGCATAACTTTGTGGCGAACGATATTATTGCG
CATAACTCTGGCCAGGGGGACAGTCTTCACGAGCACATCGCTAATCTTGC
AGGTAGCCCAGCTATCAAAAAGGGAATACTGCAGACCGTTAAGGTCGTGG
ATGAACTCGTCAAAGTAATGGGAAGGCATAAGCCCGAGAATATCGTTATC
GAGATGGCCCGAGAGAACCAAACTACCCAGAAGGGACAGAAGAACAGTAG
GGAAAGGATGAAGAGGATTGAAGAGGGTATAAAAGAACTGGGGTCCCAAA
TCCTTAAGGAACACCCAGTTGAAAACACCCAGCTTCAGAATGAGAAGCTC
TACCTGTACTACCTGCAGAACGGCAGGGACATGTACGTGGATCAGGAACT
GGACATCAATCGGCTCTCCGACTACGACGTGGATCATATCGTGCCCCAGT
CTTTTCTCAAAGATGATTCTATTGATAATAAAGTGTTGACAAGATCCGAT
AAAAATAGAGGGAAGAGTGATAACGTCCCCTCAGAAGAAGTTGTCAAGAA
AATGAAAAATTATTGGCGGCAGCTGCTGAACGCCAAACTGATCACACAAC
GGAAGTTCGATAATCTGACTAAGGCTGAACGAGGTGGCCTGTCTGAGTTG
GATAAAGCCGGCTTCATCAAAAGGCAGCTTGTTGAGACACGCCAGATCAC
CAAGCACGTGGCCCAAATTCTCGATTCACGCATGAACACCAAGTACGATG
AAAATGACAAACTGATTCGAGAGGTGAAAGTTATTACTCTGAAGTCTAAG
CTGGTCTCAGATTTCAGAAAGGACTTTCAGTTTTATAAGGTGAGAGAGAT
CAACAATTACCACCATGCGCATGATGCCTACCTGAATGCAGTGGTAGGCA
CTGCACTTATCAAAAAATATCCCAAGCTTGAATCTGAATTTGTTTACGGA
GACTATAAAGTGTACGATGTTAGGAAAATGATCGCAAAGTCTGAGCAGGA
AATAGGCAAGGCCACCGCTAAGTACTTCTTTTACAGCAATATTATGAATT
TTTTCAAGACCGAGATTACACTGGCCAATGGAGAGATTCGGAAGCGACCA
CTTATCGAAACAAACGGAGAAACAGGAGAAATCGTGTGGGACAAGGGTAG
GGATTTCGCGACAGTCCGGAAGGTCCTGTCCATGCCGCAGGTGAACATCG
TTAAAAAGACCGAAGTACAGACCGGAGGCTTCTCCAAGGAAAGTATCCTC
CCGAAAAGGAACAGCGACAAGCTGATCGCACGCAAAAAAGATTGGGACCC
CAAGAAATACGGCGGATTCGATTCTCCTACAGTCGCTTACAGTGTACTGG
TTGTGGCCAAAGTGGAGAAAGGGAAGTCTAAAAAACTCAAAAGCGTCAAG
GAACTGCTGGGCATCACAATCATGGAGCGATCAAGCTTCGAAAAAAACCC
CATCGACTTTCTCGAGGCGAAAGGATATAAAGAGGTCAAAAAAGACCTCA
TCATTAAGCTTCCCAAGTACTCTCTCTTTGAGCTTGAAAACGGCCGGAAA
CGAATGCTCGCTAGTGCGGGCGAGCTGCAGAAAGGTAACGAGCTGGCACT
GCCCTCTAAATACGTTAATTTCTTGTATCTGGCCAGCCACTATGAAAAGC
TCAAAGGGTCTCCCGAAGATAATGAGCAGAAGCAGCTGTTCGTGGAACAA
CACAAACACTACCTTGATGAGATCATCGAGCAAATAAGCGAATTCTCCAA
AAGAGTGATCCTCGCCGACGCTAACCTCGATAAGGTGCTTTCTGCTTACA
ATAAGCACAGGGATAAGCCCATCAGGGAGCAGGCAGAAAACATTATCCAC
TTGTTTACTCTGACCAACTTGGGCGCGCCTGCAGCCTTCAAGTACTTCGA
CACCACCATAGACAGAAAGCGGTACACCTCTACAAAGGAGGTCCTGGACG
CCACACTGATTCATCAGTCAATTACGGGGCTCTATGAAACAAGAATCGAC
CTCTCTCAGCTCGGTGGAGACAGCAGGGCTGACCCCAAGAAGAAGAGGAA
GGTGTCTCGAGCTGGATCCGGAGCCACGAACTTCTCTCTGTTAAAGCAAG
CAGGGGACGTGGAAGAAAACCCCGGTCCTATGCCCGCCATGAAGATCGAG
TGCCGCATCACCGGCACCCTGAACGGCGTGGAGTTCGAGCTGGTGGGCGG
CGGAGAGGGCACCCCCGAGCAGGGCCGCATGACCAACAAGATGAAGAGCA
CCAAAGGCGCCCTGACCTTCAGCCCCTACCTGCTGAGCCACGTGATGGGC
TACGGCTTCTACCACTTCGGCACCTACCCCAGCGGCTACGAGAACCCCTT
CCTGCACGCCATCAACAACGGCGGCTACACCAACACCCGCATCGAGAAGT
ACGAGGACGGCGGCGTGCTGCACGTGAGCTTCAGCTACCGCTACGAGGCC
GGCCGCGTGATCGGCGACTTCAAGGTGGTGGGCACCGGCTTCCCCGAGGA
CAGCGTGATCTTCACCGACAAGATCATCCGCAGCAACGCCACCGTGGAGC
ACCTGCACCCCATGGGCGATAACGTGCTGGTGGGCAGCTTCGCCCGCACC
TTCAGCCTGCGCGACGGCGGCTACTACAGCTTCGTGGTGGACAGCCACAT
GCACTTCAAGAGCGCCATCCACCCCAGCATCCTGCAGAACGGGGGCCCCA
TGTTCGCCTTCCGCCGCGTGGAGGAGCTGCACAGCAACACCGAGCTGGGC
ATCGTGGAGTACCAGCACGCCTTCAAGACCCCCATCGCCTTCGCCAGATC
TCGAGCTCGATGA

### Example II

### AAV Packaging and Purification

AAV were packaged via the triple-transfection method. See Zolotukhin, S., et al., Recombinant adeno-associated virus purification using novel methods improves infectious titer and yield. Gene Ther, 1999. 6(6): p. 973-85, Grieger, J. C., V. W. Choi, and R. J. Samulski, Production and characterization of adeno-associated viral vectors. Nat Protoc, 2006. 1(3): p. 1412-28, and Lock, M., et al., Rapid, simple, and versatile manufacturing of recombinant adeno-associated viral vectors at scale. Hum Gene Ther, 2010. 21(10): p. 1259-71. 293AAV cells (Cell Biolabs) were plated in growth media (DMEM+glutaMAX+pyruvate+10% FBS+1×NEAA) 1-2 days before transfection, so that confluency at transfection is between 70-90%. Media was replaced with fresh pre-warmed growth media before transfection. For each 15-cm dish, 20 ug of pHelper (Cell Biolabs), 10 ug of pRepCap [encoding capsid proteins for AAV-DJ (Cell Biolabs) or AAV9 (Penn Vector Core)], and 10 ug of pAAV were mixed in 500 ul of DMEM, and 200 ug of PEI “MAX” (Polysciences)(40 kDa, 1 mg/ml in H₂O, adjusted to pH 7.1) added for PEI:DNA mass ratio of 5:1. The mixture was incubated in the tissue culture hood for 15 mins, and transferred drop-wise to the 293AAV cell media. For large-scale AAV production, HYPERFlask ‘M’ (Corning) were used, and the transfection mixture consisted of 200 ug of pHelper, 100 ug of pRepCap, 100 ug of pAAV, and 2 mg of PEIMAX. The next day post-transfection, media was changed to DMEM+glutamax+pyruvate+2% FBS, and further incubated for 1-2 days. Cells were harvested 48-72 hrs post transfection by scrapping or dissociation with 1×PBS (pH7.2)+5 mM EDTA, and pelleted at 1500 g for 12 mins. Cell pellets were then resuspended in 1-5 ml of lysis buffer (TrisHCl pH7.5+2 mM MgCl+150 mM NaCl), and freeze-thawed 3 times between dry-ice-ethanol bath and 37° C. water bath. Cell debris was clarified via 4000 g for 5 minutes, and supernatant collected. Downstream processing differed depending on applications.

For preparation of AAV-containing lysates, the collected supernatant was first treated with 50 U/ml of Benzonase (Sigma-Aldrich) and 1 U/ml of Riboshredder (Epicentre) for 30 mins at 37° C. to remove unpackaged nucleic acids, filtered through a 0.45 um PVDF filter (Millipore), and used directly on cells or stored in −80° C.

For purification of AAV via chloroform-ammonium sulfate precipitation, 1/10^{th} volume of chloroform and NaCl (1M final concentration) was added to the lysate and shaken vigorously at room temperature. Precipitated cell debris were removed by centrifugation (4000 g 30 mins), and supernatant collected. PEG-8000 (10% final w/v) was added to the supernatant, and incubated on ice for at least 1 hr or overnight. PEG-precipitated virions were then collected via centrifugation (4000 g 30 mins 4° C.), and resuspended in 50 mM HEPES buffer (pH8). 50 U/ml of Benzonase (Sigma-Aldrich) and 1 U/ml of Riboshredder (Epicentre) were added and incubated for 30 mins at 37° C. An equal volume of chloroform was then added, and vigorously vortexed. The precipitate was removed from the aqueous phase via centrifugation, and the aqueous phase collected and allowed to stand for 30 mins in the tissue culture hood for residual chloroform to evaporate. Ammonium sulfate was added to 0.5M, chilled on ice for at least 1 hour, centrifuged at 4000 g for 30 mins, and the supernatant collected. Additional ammonium sulfate was then added to 2M, chilled on ice overnight, and the precipitated virions collected via 4000 g 30 mins 4° C. AAVs were then resuspended and dialyzed in 1×PBS+35 mM NaCl, quantified for titers, and stored in −80° C.

For purification of AAV via iodixanol density gradient ultracentrifugation (see Zolotukhin, S., et al., Recombinant adeno-associated virus purification using novel methods improves infectious titer and yield. Gene Ther, 1999. 6(6): p. 973-85, and Grieger, J. C., V. W. Choi, and R. J. Samulski, Production and characterization of adeno-associated viral vectors. Nat Protoc, 2006. 1(3): p. 1412-28), the collected supernatant was first treated with 50 U/ml Benzonase and 1 U/ml Riboshredder for 30 mins at 37° C. After incubation, the lysate was concentrated to <3 ml by ultrafiltration with Amicon Ultra-15 (50 kDa MWCO)(Millipore), and loaded on top of a discontinuous density gradient consisting of 2 ml each of 15%, 25%, 40%, 60% Optiprep (Sigma-Aldrich) in an 11.2 ml Optiseal polypropylene tube (Beckman-Coulter). The volumes were topped up with lysis buffer. The tubes were centrifuged at 58000 rpm, at 18° C., for 1.5 hrs, on an NVT65 rotor. The 40% fraction was then extracted via a 18G needle, and dialyzed with 1×PBS (pH 7.2) supplemented with 35 mM NaCl, using Amicon Ultra-15 (50 kDa or 100 kDa MWCO)(Millipore). The purified AAVs were then quantified for titers, and stored in −80° C.

AAV titers (vector genomes) were quantified via hydrolysis-probe-based qPCR (see Aurnhammer, C., et al., Universal real-time PCR for the detection and quantification of adeno-associated virus serotype 2-derived inverted terminal repeat sequences. Hum Gene Ther Methods, 2012. 23(1): p. 18-28) against standard curves generated from linearized parental AAV plasmids and rAAV2RSM (ATCC VR-1616).

qPCR Probes and Primers:

| Target locus | Sequence |
|---|---|
| AAV ITR F | GGAACCCCTAGTGATGGAGTT |
| | (SEQ ID NO: 37) |
| AAV ITR R | CGGCCTCAGTGAGCGA |
| | (SEQ ID NO: 38) |
| AAV ITR probe | /56-FAM/CACTCCCTCTCTGCGCGCTCG/3BH |
| | (SEQ ID NO: 39) |

### Example III

### Cell Culture Transfection and Transduction

All cells were incubated at 37° C. and 5% CO₂. C2C12 cells were grown in growth media (DMEM+glutaMAX+10% FBS). Cells were split with TypLE Express (Invitrogen) every 2-3 days before confluency of 80% is reached to prevent terminal differentiation. Passage number was kept below 15 for all experiments. For transfection of C2C12 myoblasts, 10⁵ cells were plated per well in a 24-well plate, in 500 ul of growth media. The following day, the media was replaced with fresh growth media, and 800 ng of total plasmid DNA was transfected with 2.4 ul of Lipofectamine 2000 (Life Technologies) according to manufacturer's protocol. 1:1 mass ratio of vectors encoding Cas9:gRNA(s) was used. Media was replaced with differentiation media (DMEM+glutaMAX+2% DHS) on the 1^{st} and 3^{rd} days post-lipofection.

For differentiation of C2C12 into myotubes, 2×10⁴ cells were plated per well in a 96-well plate, in 100 ul of growth media. At confluency 1-2 day(s) post-plating, media was replaced with fresh differentiation media, and further incubated for 4-5 days. Fresh differentiation media was replaced before transduction. For transduction with AAV-containing lysates, 50 ul of each lysate was added per well. For transduction with purified AAV, the stated titers of vector genomes (vg) were added per well. Culture media was replaced with fresh differentiation media the next day, and cells were incubated for stated durations.

For puromycin selection, 3 ug of puromycin dihydrochloride (Sigma) per ml of cell culture media was applied 2 days post-transfection. 100 nM of dexamethasone (Sigma) was used for C2C12 atrophy experiments.

The LSL-tdTomato reporter cell line was derived from tail-tip fibroblasts of the Ai9 mouse strain (JAX 007909), and immortalized with lentiviruses encoding the large SV40 T-antigen. Cells were cultured in DMEM+pyruvate+glutaMAX+10% FBS. For transduction with AAVs, cells were plated at 2×10⁴ per well in a 96-well plate, in 100 ul of growth media. AAV-containing lysates or purified AAVs were applied at confluency of 70-90%. Culture media was replaced with fresh growth media the next day, and cells were incubated for stated durations.

The GC-1 spg mouse spermatogonial cell line (CRL-2053) was obtained from ATCC, and cultured in DMEM+pyruvate+glutaMAX+10% FBS. Transduction of the cells with AAV was performed similarly to the LSL-tdTomato cell line.

### Example IV

### Genotyping and Analysis

C2C12 cells were harvested 4 days post-lipofection, with 100 ul of QuickExtract DNA Extraction Solution (Epicentre) per well of a 24-well plate; and C2C12 myotubes were harvested 7 days post-transduction, with 20 ul of DNA QuickExtract per well of a 96-well plate. The cell lysates were heated at 65° C. for 10 mins, 95° C. for 8 mins, and stored at −20° C. Each locus was amplified from 0.25-0.5 ul of cell culture lysate per 25 ul PCR reaction, for 15-25 cycles, to minimize amplification bias.

For barcoding for deep sequencing, 1 ul of each unpurified PCR reaction was added to 25 ul of barcoding PCR reaction, and thermocycled for 10 cycles. Amplicons were pooled, and the whole sequencing library was purified with home-made SPRI beads (9% PEG8000 final concentration), and sequenced on a Miseq (Illumina) for 2×251 cycles. FASTQ were analyzed with BLAT (with parameters -t=dna -q=dna -tileSize=11 -stepSize=5 -oneOff=1 -repMatch=10000000 -minMatch=4 -minIdentity=90 -maxGap=3 -noHead) and post-alignment analyses performed with custom MATLAB (MathWorks) scripts. Alignments due to primer dimers were excluded by filtering off sequence alignments that do not extend >2 bp into the targeted loci from the locus-specific primers. To minimize the impact of sequencing errors, conservative variant calling was performed by ignoring base substitutions, and considering only variants that overlap with a ±30 bp window from the designed CRISPR cut sites. Cas9-only controls were similarly analyzed.

### Example V

### AAV Administration in Mice

For neonatal systemic injection (see Foust, K. D., et al., Intravascular AAV9 preferentially targets neonatal neurons and adult astrocytes. Nat Biotechnol, 2009. 27(1): p. 59-65, Bostick, B., et al., Systemic AAV-9 transduction in mice is influenced by animal age but not by the route of administration. Gene Ther, 2007. 14(22): p. 1605-9, Byrne, L. C., et al., The Expression Pattern of Systemically Injected AAV9 in the Developing Mouse Retina Is Determined by Age. Mol Ther, 2014, Gombash Lampe, S. E., B. K. Kaspar, and K. D. Foust, Intravenous injections in neonatal mice. J Vis Exp, 2014(93), and Sands, M. S. and J. E. Barker, Percutaneous intravenous injection in neonatal mice. Lab Anim Sci, 1999. 49(3): p. 328-30), the mother was first removed from the cage, and 1 to 4-day old neonates were individually placed on ice for sedation. For intravenous injections, 1×10¹¹ to 5×10¹¹ vector genomes (vg) of total AAV9 were injected into each neonate via the superficial temporal vein using a 3/10 cc 30G insulin syringe. Higher dosages between 5×10¹¹ to 5×10¹³ can be used. Same dosages of viruses were utilized for intraperitoneal injections. Vector volumes were kept below 50 ul, alternatively, volumes of 50 ul or more are acceptable. Injected neonates were gently cleaned, replaced into the cage, and rubbed with bedding. The mother was then returned to the cage after nose-numbing with ethanol pads.

For systemic injection into adult male or pregnant (E16) female mice, mice were anesthetized with isoflurane and the tail was swabbed with ethanol before being placed under a heated lamp. 1×10¹² to 6×10¹² vg of AAV9 were injected through the tail vein. The injected volume was kept under 150 ul. Higher dosages between 5×10¹¹ to 5×10¹⁵ can be used. Also, calibration to body weight is desirable depending on the animal or patient size, for example 10¹⁰ to 10¹⁷ per kg of body weight.

Animals were sacrificed via CO₂ asphyxia 2, 4, 8, or 10 weeks following injections. Histology was performed for the skeletal muscles (tibialis anterior, extensor digitorum longus, gastronomies, and quadriceps), heart, liver, diaphragm, brain, and gonads.

### Example VI

### Mouse Breeding Experiments

Male mice injected with AAV as neonates were allowed to reproductively mature (˜1 month of age), and crossed to uninjected Ai9 female mice. Male mice injected as adults were crossed to uninjected Ai9 female mice following AAV administration. Female mice were rotated weekly for multiple crosses, and also for tracking persistence of genetic modifications within spermatogonial stem cells.

### Example VII

### Imaging and Analyses

Confocal images were taken using a Zeiss LSM780 inverted microscope. For live cell-imaging, each image consists of 3× z-stacks (7 um intervals) and 2×2 tiles. For fixed histological samples, the number of z-stacks and tiles are indicated in the figure legends. Fluorescent images were merged by maximum intensity projection. All images were then analyzed via Cellprofiler (see Carpenter, A. E., et al., CellProfiler: image analysis software for identifying and quantifying cell phenotypes. Genome Biol, 2006. 7(10): p. R100) and custom MATLAB (MathWorks) scripts.

### Example VIII

### Expression and Linking of Portions of Cas9

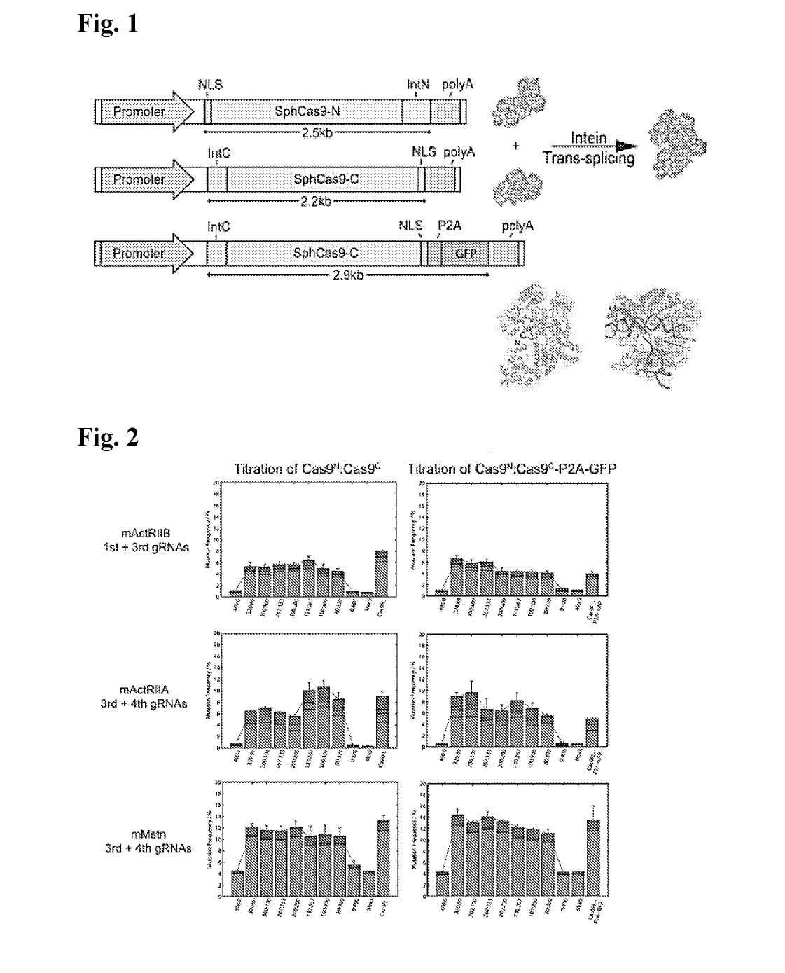

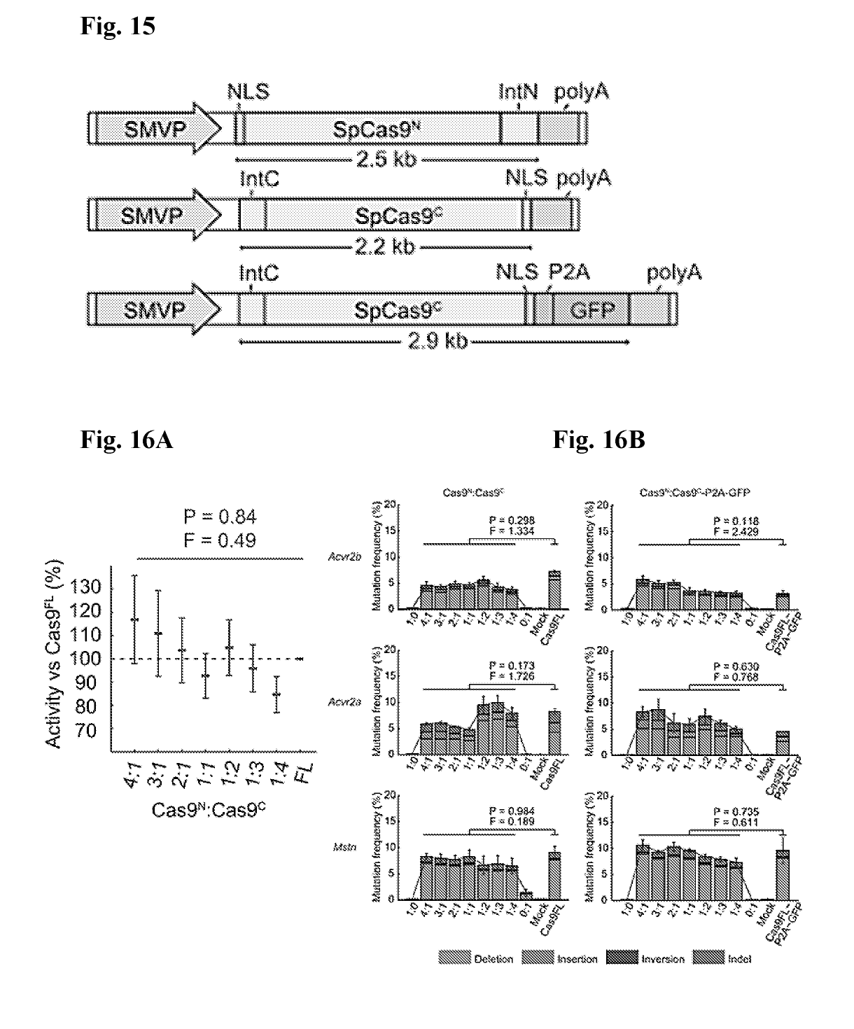

The Sp Cas9 coding sequence is about 4.2 kb in length. The Sp Cas9 protein consists of a bi-lobed structure, with a disordered linker between amino acid residues V713 and D718. See Nishimasu, H., et al., Crystal structure of Cas9 in complex with guide RNA and target DNA. Cell, 2014. 156(5): p. 935-49 and Jinek, M. et al. Structures of Cas9 endonucleases reveal RNA-mediated conformational activation. Science, 2014, 343(6176): p. 1247997-1247997. Each lobe may be thought of as a portion of the Sp Cas9. The Sp cas9 has a first portion and a second portion, which if separate, can be linked together to form the Sp Cas9. This may be referred to herein as a split Cas9 design. According to one aspect, the first portion or sequence includes or has the Cas9 sequence up to and including V713. The second portion or sequence begins with S714 and includes the remaining portion or sequence of Cas9. According to the methods provided herein, each lobe is separately expressed and folded, and reconstituted in vivo by split-intein protein trans-splicing. See Li, J., et al., Protein trans-splicing as a means for viral vector-mediated in vivo gene therapy. Hum Gene Ther, 2008. 19(9): p. 958-64, Wu, H., Z. Hu, and X. Q. Liu, Protein trans-splicing by a split intein encoded in a split DnaE gene of Synechocystis sp. PCC6803. Proc Natl Acad Sci USA, 1998. 95(16): p. 9226-31, and Lohmueller, J. J., T. Z. Armel, and P. A. Silver, A tunable zinc finger-based framework for Boolean logic computation in mammalian cells. Nucleic Acids Res, 2012. 40(11): p. 5180-7. The N-terminal lobe is designed to be fused on its C-terminus with the Rhodothermus marinus N-split-intein (Cas9^{N}), and the C-terminal lobe with C-split-intein (Cas9C). This reduces the coding sequences to 2.5 kb and 2.2 kb respectively, and providing >2 kb within each AAV for incorporation of transcriptional elements, gRNAs and fusion domains. See FIG. 1 and FIG. 15.

The split-Cas9 design was tested by individually targeting three genes involved in muscular growth inhibition (mMstn, mActRIIB, mActRIIA). See McPherron, A. C. and S. J. Lee, Double muscling in cattle due to mutations in the myostatin gene. Proc Natl Acad Sci USA, 1997. 94(23): p. 12457-61, Lee, S. J. and A. C. McPherron, Regulation of myostatin activity and muscle growth. Proc Natl Acad Sci USA, 2001. 98(16): p. 9306-11, Lee, S. J., Regulation of muscle mass by myostatin. Annu Rev Cell Dev Biol, 2004. 20: p. 61-86, Williams, M. S., Myostatin mutation associated with gross muscle hypertrophy in a child. N Engl J Med, 2004. 351(10): p. 1030-1; author reply 1030-1, Lee, S. J., et al., Regulation of muscle growth by multiple ligands signaling through activin type II receptors. Proc Natl Acad Sci USA, 2005. 102(50): p. 18117-22, and Lee, S. J., et al., Regulation of muscle mass by follistatin and activins. Mol Endocrinol, 2010. 24(10): p. 1998-2008. Plasmids encoding split-Cas9 (i.e., a plasmid encoding a first portion of the Sp Cas9 and a plasmid encoding a second portion of the Cas9, wherein the first portion and the second portion when linked together form the Sp Cas9) and gRNAs were transfected into proliferating C2C12 myoblasts with Cas9^{N}:Cas9^{C} ranging from 1:0 to 0:1 as shown in FIG. 2, keeping total plasmid amount constant, and mutagenic endonucleolytic-activity was compared against SphCas9^{FL}. The reconstituted split-Cas9 retained full biological activity, as determined by similar induced mutational frequencies compared to SphCas9^{FL}. Furthermore, the working ratio of Cas9^{N}:Cas9^{C} spans the entire tested range of 4:1 to 1:4, demonstrating that effective expression of Cas9 is not limiting. Expression of both the first portion and the second portion is necessary for activity, since expressing each portion in isolation (1:0 or 0:1) does not reconstitute CRISPR activity.

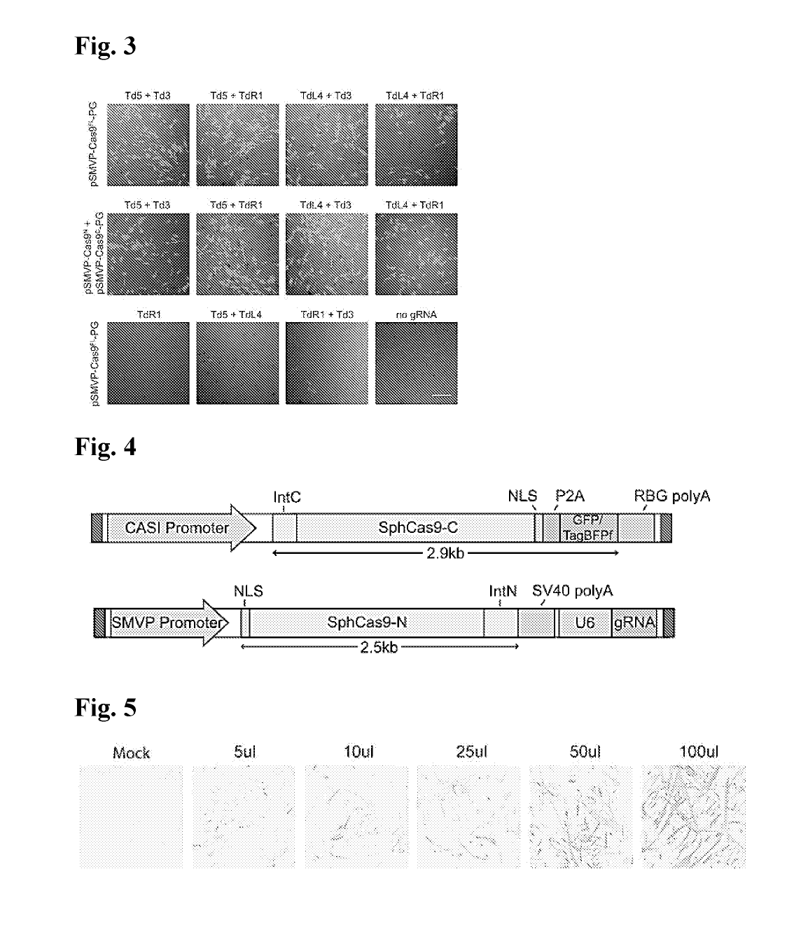

CRISPR-mediated excision rates on a single-cell level, using the Ai9 excision-activated-tdTomato reporter fibroblasts as previously described were examined to compare efficiencies of split-Cas9 and full-length Cas9. Split-Cas9 and full-length Cas9 led to similar numbers of edited cells, across all four gRNA pairs tested as shown in FIG. 3. GFP fluorescence intensity of the co-translating Cas9-P2A-turboGFP was close to background levels in most cells, which suggests that low expression levels of Cas9 can be sufficient to induce endonucleolytic activity.

The split-Cas9 and gRNAs were packaged into AAV-DJs, a hybrid serotype evolved through capsid shuffling. See Grimm, D., et al., In vitro and in vivo gene therapy vector evolution via multispecies interbreeding and retargeting of adeno-associated viruses. J Virol, 2008. 82(12): p. 5887-911. AAVs were generated at high titers, as expected from the usage of optimal genome sizes. Cas9^{C} expression from AAV transduction was detected via co-translating P2A-turboGFP. Since the majority of somatic tissues in adult organisms are terminally differentiated, transduction with differentiated C2C12 mouse myotubes was performed. FIG. 4 is a schematic of the AAV-CRISPR constructs. Cassettes are flanked by AAV ITRs shown in dark gray. FIG. 5 shows that transduction efficiency, as detected via P2A-turboGFP, is correlated with the amount of AAV-containing lysate added to the differentiated C2C12 myotubes.

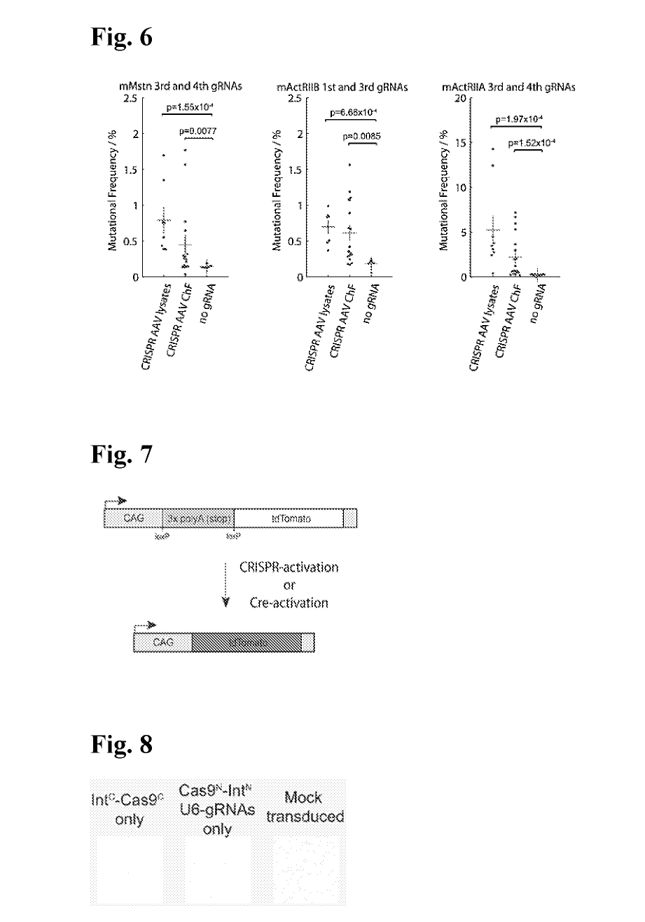

AAV-DJs encoding CRISPR targeting mMstn, mActRIIB, mActRIIA, and the LSL-tdTomato reporter were generated. Cells were transduced cells with the viruses and assayed for mutational activity and fluorescence-activation. A total of 100 ul of AAV-containing lysates or 10¹⁰ vector genomes (vg) of chloroform-ammonium sulfate purified AAVs were used. AAV-CRISPR induced on-target mutations on all three endogenous genes as shown in FIG. 6. AAV-CRISPR also activated the LSL-tdTomato reporter. See FIG. 7, FIG. 8 and FIG. 9.

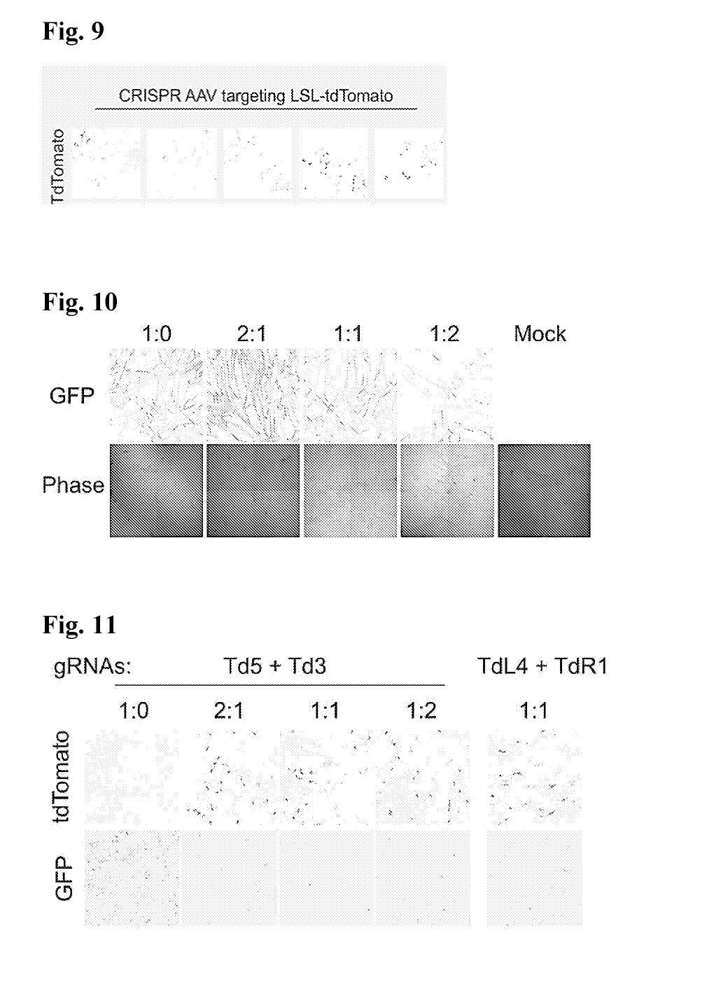

Because elimination of empty capsids from the AAV preparations can result in enhanced transduction efficiency and reduced immunological burden in animals (see Gao, K., et al., Empty Virions In AAV8 Vector Preparations Reduce Transduction Efficiency And May Cause Total Viral Particle Dose-Limiting Side-Effects. Mol Ther Methods Clin Dev, 2014. 1(9): p. 20139, Ayuso, E., et al., High AAV vector purity results in serotype- and tissue-independent enhancement of transduction efficiency. Gene Ther, 2010. 17(4): p. 503-10, and Zeltner, N., et al., Near-perfect infectivity of wild-type AAV as benchmark for infectivity of recombinant AAV vectors. Gene Ther, 2010. 17(7): p. 872-9), subsequent experiments were carried out with fully-packaged virions purified via density gradient ultracentrifugation. See Zolotukhin, S., et al., Recombinant adeno-associated virus purification using novel methods improves infectious titer and yield. Gene Ther, 1999. 6(6): p. 973-85, and Grieger, J. C., V. W. Choi, and R. J. Samulski, Production and characterization of adeno-associated viral vectors. Nat Protoc, 2006. 1(3): p. 1412-28. A total of 1×10¹² (vg) AAV-DJ-CRISPR targeting the myostatin gene in C2C12 myotubes were transduced, while varying the ratios of Cas9^{N}:Cas9c. See FIG. 10. Similarly, 1×10¹² AAV-DJ-CRISPR activating the LSL-tdTomato reporter cell line were transduced. See FIG. 11.

Split-Cas9 and gRNAs are packaged into AAV9, a serotype that exhibits broad systemic transduction in mammals, with tropism preference to cardiac and skeletal muscles, and robust transduction of other organs such as the liver, nervous system, lungs, kidneys, spleen, and gonads. See Zincarelli, C., et al., Analysis of AAV serotypes 1-9 mediated gene expression and tropism in mice after systemic injection. Mol Ther, 2008. 16(6): p. 1073-80, Inagaki, K., et al., Robust systemic transduction with AAV9 vectors in mice: efficient global cardiac gene transfer superior to that of AAV8. Mol Ther, 2006. 14(1): p. 45-53, Keeler, A. M., et al., Long-term correction of very long-chain acyl-coA dehydrogenase deficiency in mice using AAV9 gene therapy. Mol Ther, 2012. 20(6): p. 1131-8, Gray, S. J., et al., Preclinical differences of intravascular AAV9 delivery to neurons and glia: a comparative study of adult mice and nonhuman primates. Mol Ther, 2011. 19(6): p. 1058-69, Okada, H., et al., Robust Long-term Transduction of Common Marmoset Neuromuscular Tissue With rAAV1 and rAAV9. Mol Ther Nucleic Acids, 2013. 2: p. e95, and Foust, K. D., et al., Intravascular AAV9 preferentially targets neonatal neurons and adult astrocytes. Nat Biotechnol, 2009. 27(1): p. 59-65. Within the skeletal muscles, AAV9 exhibits transduction preference for fast-twitch myofibers (see Bostick, B., et al., Systemic AAV-9 transduction in mice is influenced by animal age but not by the route of administration. Gene Ther, 2007. 14(22): p. 1605-9), which corresponds to the predominant myostatin-expressing fiber type. See Carlson, C. J., F. W. Booth, and S. E. Gordon, Skeletal muscle myostatin mRNA expression is fiber-type specific and increases during hindlimb unloading. Am J Physiol, 1999. 277(2 Pt 2): p. R601-6. The biodistribution of CRISPR activity following systemic injection of AAV9-CRISPR in neonatal and adult mice may be determined. The mMstn gene is targeted to induce muscle hypertrophy, and the LSL-tdTomato locus is targeted for unbiased fluorescent detection of CRISPR activity. A functional CRISPR system using a split Cas9 design can be delivered systemically into a subject such as a plant or animal.

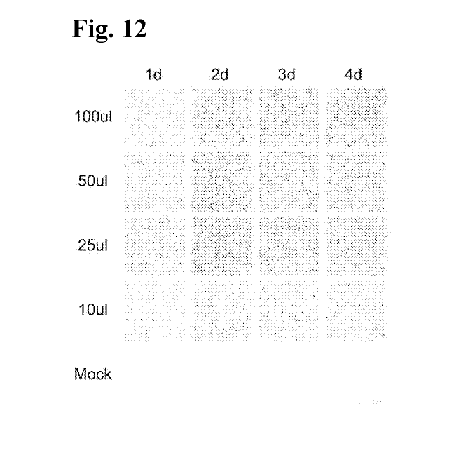

According to certain aspects, AAV9-CRISPR is injected systemically into neonatal and adult male mice to produce genetic modifications that are subsequently vertically transmitted. Spermatogenesis begins from the type A spermatogonial primitive stem cells (A_{undiff}), which give rise to all differentiating spermatogonia, spermatocytes, and spermatids that progressively migrate through a dynamic Sertoli cell layer into the seminiferous tubule lumen. See Yoshida, S., M. Sukeno, and Y. Nabeshima, A vasculature-associated niche for undifferentiated spermatogonia in the mouse testis. Science, 2007. 317(5845): p. 1722-6. The A_{undiff} spermatogonial stem cells reside between the vasculature and the blood-testis barrier as defined by Sertoli cell-Sertoli cell tight junctions, specifically in a microenvironment niche around vascular branchpoints (see Yoshida, S., M. Sukeno, and Y. Nabeshima, A vasculature-associated niche for undifferentiated spermatogonia in the mouse testis. Science, 2007. 317(5845): p. 1722-6), suggesting that these progenitors might be exposed to intravascularly delivered AAVs. As shown in FIG. 12, GC-1 spermatogonial stem cells (see Hofmann, M. C., et al., Immortalization of germ cells and somatic testicular cells using the SV40 large T antigen. Exp Cell Res, 1992. 201(2): p. 417-35) are permissive to AAV transduction suggesting that intravascularly delivered AAVs can likewise transduce the spermatogonia in whole mammals.

AAV9-CRISPR is injected systematically or locally into neonatal and adult male mice to target the LSL-tdTomato reporter and the myostatin gene. Genotype, transduction efficiency, fluorescence activation by CRISPR, body mass growth curve, serum myostatin isoforms, muscle fiber cross-sectional areas, histology of various organ types, germline transmission to F1 progeny following crosses to uninjected females is analyzed.

### Example IX

### Multiplexing AAV-CRISPR

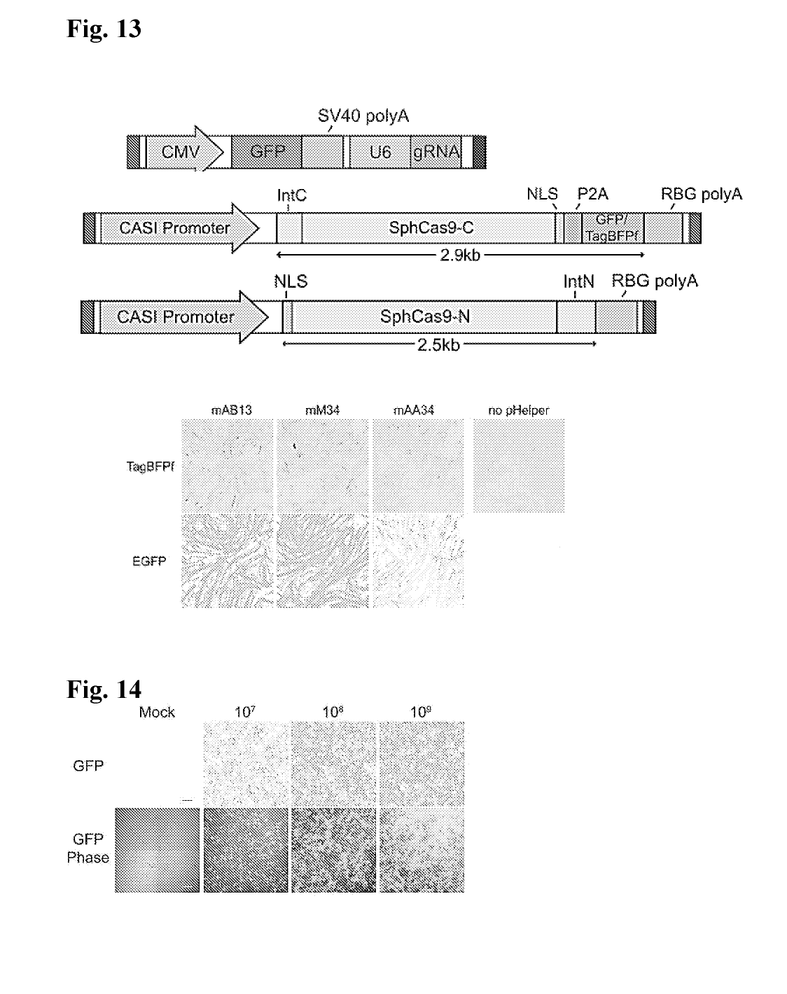

A key strength of CRISPR is its programmability, and facile multiplex gene targeting can be accomplished with a simple recoding of the gRNA spacer sequence. gRNAs can also be provided in separate AAVs, each targeting a different DNA site. The gRNAs can be encoded by self-complementary AAVs (scAAVs) (see FIG. 13 and FIG. 14), which deliver and express transgenes more efficiently than ssAAVs via bypassing the rate-limiting step of second-strand synthesis. See McCarty, D. M., P. E. Monahan, and R. J. Samulski, Self-complementary recombinant adeno-associated virus (scAAV) vectors promote efficient transduction independently of DNA synthesis. Gene Ther, 2001. 8(16): p. 1248-54 and McCarty, D. M., et al., Adeno-associated virus terminal repeat (TR) mutant generates self-complementary vectors to overcome the rate-limiting step to transduction in vivo. Gene Ther, 2003. 10(26): p. 2112-8. Further, split-Cas9 architecture reduces the coding sequences dramatically. The length of the coding sequences are close to the packaging limit of scAAVs (commonly thought as 2.2 kb-2.4 kb, although reported to be ˜3.3 kb (see Wu, Jianqing, et al. Self-complementary recombinant adeno-associated viral vectors: packaging capacity and the role of rep proteins in vector purity. Human gene therapy, 2007. 18(2): p. 171-182), which transduce cells and animals dramatically better than conventional single-strand AAVs. Packaging of all components of CRISPR into scAAVs for delivery will enable significantly more robust gene-editing efficiencies, even at lower dosages.

### Example X

### Alternatives to Split Cas9 for AAV Packaging

Various shorter promoters, Cas9 transgenes, and polyA sequences were tested. For the promoter sequence, the 173 bp truncated mCMV promoter (see Ostedgaard, L. S., et al., A shortened adeno-associated virus expression cassette for CFTR gene transfer to cystic fibrosis airway epithelia. Proc Natl Acad Sci USA, 2005. 102(8): p. 2952-7), 312 bp synthetic muscle-specific promoter C5-12 (see Wang, B., et al., Construction and analysis of compact muscle-specific promoters for AAV vectors. Gene Ther, 2008. 15(22): p. 1489-99), 376 bp Tre3G promoter (7×TetO)(Clontech), and a truncated 231 bp Tre3G^{tran} promoter (3×TetO) were tested. Notably, the Tre3G promoter offers tight temporal control of Cas9 expression by doxycycline induction, while allowing Cas9 expression to be dependent on a larger and more regulated tissue-specific promoter via trans-activation (driven by tTA or rtTA). Only the Tre3G promoter expressed Cas9 well enough to achieve mutational activity comparable to the strong ubiquitous hybrid promoters. The shorter ST1 CRISPR system was also tested, but the lower mutational frequency and restrictive PAM (NNAGAA) (see Esvelt, K. M., et al., Orthogonal Cas9 proteins for RNA-guided gene regulation and editing. Nat Methods, 2013. 10(11): p. 1116-21) may make it less attractive. Truncation of the 133aa REC2 domain of Sp Cas9 abolishes targeting activity. Finally, both the SV40 polyA and the 49 bp synthetic polyA (see Levitt, N., et al., Definition of an efficient synthetic poly(A) site. Genes Dev, 1989. 3(7): p. 1019-25) resulted in similar mutational frequency when paired with strong ubiquitous hybrid promoters.

### Example XI

### Transduction of Spermatogonial Stem Cells with Purified scAAV-DJ and scAAV9 Conditioned Media

In parallel with transduction of GC-1 cells with crude and purified scAAV9, robust transduction with conditioned media from producer cells was also demonstrated. Unlike that of AAV2 variants, which are retained within the virus-producing HEK cells, serotype 9 viruses are both found within the producer cells and released into the cell culture media during production. See Lock, M., et al., Rapid, simple, and versatile manufacturing of recombinant adeno-associated viral vectors at scale. Hum Gene Ther, 2010. 21(10): p. 1259-71 and Vandenberghe, L. H., et al., Efficient serotype-dependent release of functional vector into the culture medium during adeno-associated virus manufacturing. Hum Gene Ther, 2010. 21(10): p. 1251-7. Extraction of AAV9 from culture media bypasses the need for clarification of abundant cellular debris that is associated with producer cells lysis, potentially reducing the chance for protein contaminants in the final virus preparations. Furthermore, GC-1 cells are also robustly transduced with scAAV-DJ. Spermatologocal stem cells are permissive to AAV hybrid serotype DJ. scAAV-DJ encoding CMV-EGFP-SV40polyA-U6-gRNA (mMstn3 and mMstn4) was applied to GC-1 spg spermatogonial stem cell line.

### Example XII

### Split-Cas9 Transfected as Plasmids (Lipofection)

The biological activity of split-Cas9 was investigated in transfected C2C12 myoblasts. Split-Cas9 was fully active, targeting all endogenous genes tested (Acvr2b, Acvr2a, and Mstn) at efficiencies 85% to 115% of Cas9^{FL}. See FIG. 16A and FIG. 16B. Working ratios of Cas9^{N}:Cas9^{C} spanned the entire tested range of 4:1 to 1:4, while each half in isolation failed to reconstitute CRISPR activity. Likewise, the activities of split-Cas9 and Cas9^{FL} were equivalent in Ai9 tail-tip fibroblasts, across four gRNA pairs examined. Therefore, split-intein-reconstituted split-Cas9, unlike non-covalent heterodimers (see Wright, A. V. et al. Rational design of a split-Cas9 enzyme complex. Proceedings of the National Academy of Sciences of the United States of America, 2015. 112(10): p. 2984-2989 and Zetsche, B., Volz, S. E. & Zhang, F. A split-Cas9 architecture for inducible genome editing and transcription modulation. Nature biotechnology, 2015. 33: p. 139-142), exhibits equivalent activity as Cas9^{FL}, implying that Cas9 tertiary structure and function are preserved with scarless protein ligation.

### Cell Line #1: C2C12 Myoblasts

C2C12 myoblasts C2C12 cells were obtained from the American Tissue Collection Center (ATCC, Manassas, Va.), and grown in growth media (DMEM+glutaMAX+10% FBS). Cells were split with TypLE Express (Invitrogen) every 2-3 days and before reaching 80% confluency, to prevent terminal differentiation. Passage number was kept below 15 for all experiments. For transfection of C2C12 myoblasts, 10⁵ cells were plated per well in a 24-well plate, in 500 μl of growth media. The following day, the media was replaced with fresh growth media, and 800 ng of total plasmid DNA was transfected with 2.4 μl of Lipofectamine 2000 (Life Technologies) according to manufacturer's protocol. 1:1 mass ratio of vectors encoding Cas9:gRNA(s) was used. Media was replaced with differentiation media (DMEM+glutaMAX+2% donor horse serum) on the 1^{st} and 3^{rd} days post-lipofection. C2C12 cells were harvested 4 days post-lipofection, with 100 μl of QuickExtract DNA Extraction Solution (Epicentre) per well of a 24-well plate. The cell lysates were heated at 65° C. for 10 min., 95° C. for 8 min., and stored at ˜20° C.

Each locus was amplified from 0.5 μl of cell culture lysate per 25 μl PCR reaction, for 20-25 cycles. For barcoding for deep sequencing, 1 μl of each unpurified PCR reaction was added to 20 μl of barcoding PCR reaction, and thermocycled [95° C. for 3 min., and 10 cycles of (95° C. for 10 s, 72° C. for 65 s)]. Amplicons were pooled, and the whole sequencing library was purified with self-made SPRI beads (9% PEG final concentration), and sequenced on a Miseq (Illumina) for 2×251 cycles. FASTQ were analyzed with BLAT (with parameters -t=dna -q=dna -tileSize=1 -stepSize=5 -oneOff=1 -repMatch=10000000 -minMatch=4 -minIdentity=90 -maxGap=3 -noHead) and post-alignment analyses performed with custom MATLAB (MathWorks) scripts. Alignments due to primer dimers were excluded by filtering off sequence alignments that did not extend >2 bp into the loci from the locus-specific primers. To minimize the impact of sequencing errors, conservative variant calling was performed by ignoring base substitutions, and calling only variants that overlap with a ±30 bp window from the designated CRISPR cut sites. Controls were equally analyzed.

### Locus-Specific Amplification Primers for Deep Sequencing (Black: Locus-Specific Sequences; Bold: Part of Illumina Sequencing Adaptor):

| Target | |
|---|---|
| locus | Sequence |
| Acvr2b F | CTTTCCCTACACGACGCTCTTCCGATCTNNNNNNCTGG |
| | AGTGTTAGAGTGGGCG (SEQ ID NO: 40) |
| Acvr2b R | GGAGTTCAGACGTGTGCTCTTCCGATCTGACTGCCCCA |
| | TGGAAAGACA (SEQ ID NO: 41) |
| Mstn F | CTTTCCCTACACGACGCTCTTCCGATCTNNNNNNGGGC |
| | CATGAAAGGAAAAATGAAGT (SEQ ID NO: 42) |
| Mstn R | GGAGTTCAGACGTGTGCTCTTCCGATCTGCCTCTGGGG |
| | TTTGCTTGGT (SEQ ID NO: 43) |
| Acvr2a F | CTTTCCCTACACGACGCTCTTCCGATCTNNNNNNGAGA |
| | TATAAGCTGAATAAGGCCAATGACATACT (SEQ ID |
| | NO: 44) |
| Acvr2a R | GGAGTTCAGACGTGTGCTCTTCCGATCTCTACTGCTCT |
| | TTCCTGCCGA (SEQ ID NO: 45) |

gRNA Spacer Sequences:

| | Spacer sequence, including 5′ G |
|---|---|
| Sp gRNAs | U6 promoter |
| Acw2b B1 | GGGCCATGTGGACATCCATGAGGTGAGACAGTGCCAGC |
| | GT (SEQ ID NO: 46) |
| Acw2b B3 | GGCCTGAAGCCACTACAGCTGCTGGAGATCAAGGCTCG |
| | (SEQ ID NO: 47) |
| Acw2a A3 | GGCCCTAGCATCTAAGTTCTCGCAGGC |
| | (SEQ ID NO: 48) |
| Acw2a A4 | GGTCATTCCATCTCAGCTGTGACAGCAGCGCAGAA |
| | (SEQ ID NO: 49) |
| Mstn M3 | GTCAAGCCCAAAGTCTCTCCGGGACCTCTT |
| | (SEQ ID NO: 50) |
| Mstn M4 | GGAATCCCGGTGCTGCCGCTACCCCCTCA |
| | (SEQ ID NO: 51) |

### Cell Line #2: Ai9 Reporter Fibroblasts (Aka 3×Stop-tdTomato)

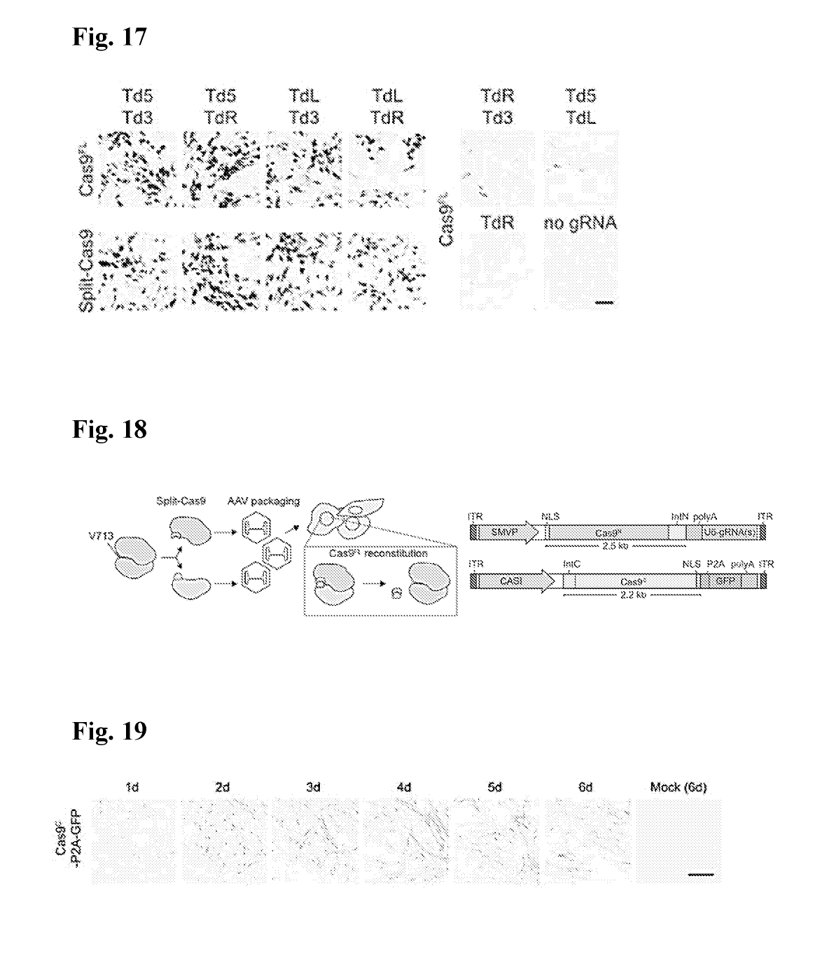

The 3×Stop-tdTomato reporter cell line was derived from tail-tip fibroblasts of Ai9 mouse, Madisen, L. et al. A robust and high-throughput Cre reporting and characterization system for the whole mouse brain. Nat Neurosci, 2010. 13: p. 133-140, (JAX 007905), and immortalized with lentiviruses encoding the large SV40 T-antigen (GenTarget Inc, LVP016-Puro). Cells were cultured in DMEM+pyruvate+glutaMAX+10% FBS. Lipofectamine 2000 (Life Technologies) was used for transfection of plasmids, and images were taken 5 days after transfection. See FIG. 17.

gRNA Spacer Sequences:

| | Spacer sequence, including 5′ G from U6 |
|---|---|
| Sp gRNAs | promoter |
| Ai9 Td5 | GCTAGAGAATAGGAACTTCTT (SEQ ID |
| | NO: 52) |
| Ai9 TdL | GAAAGAATTGATTTGATACCG (SEQ ID |
| | NO: 53) |
| Ai9 Td3 | GATCCCCATCAAGCTGATC (SEQ ID NO: 54) |
| Ai9 TdR | GGTATGCTATACGAAGTTATT (SEQ ID |
| | NO: 55) |

### Example XIII

### Split-Cas9 Transduced as AAVs in Cell Culture

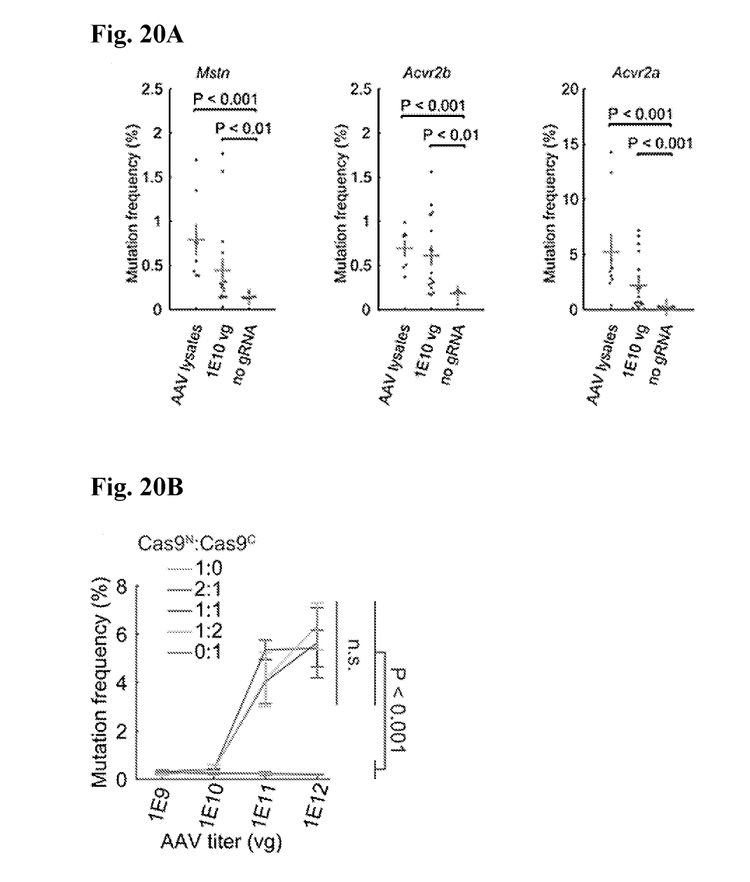

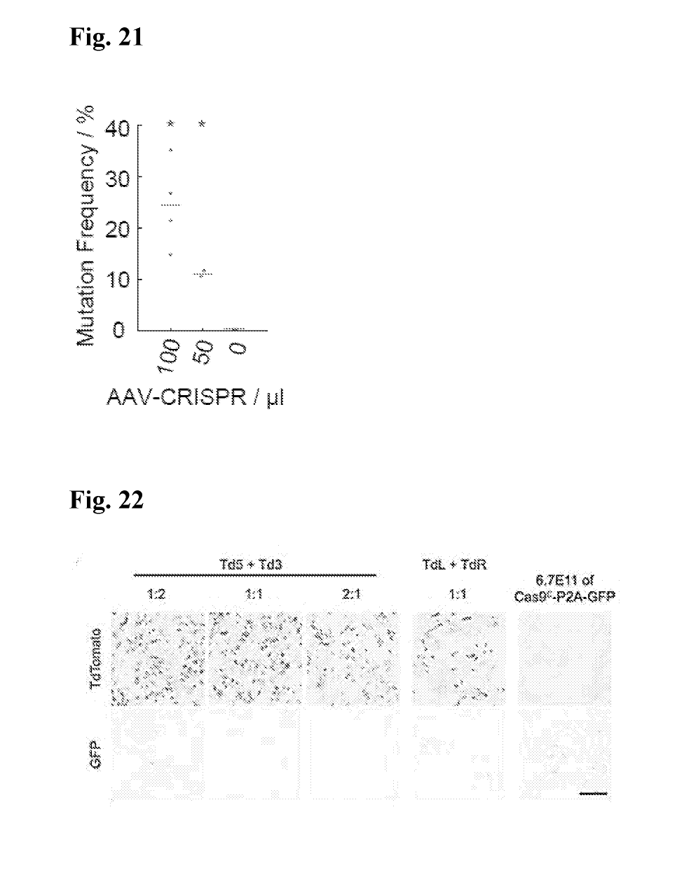

To assay the activity of split-Cas9 in terminally differentiated, post-mitotic cells, split-Cas9 and paired gRNAs (targeting Acvr2b, Acvr2a, and Mstn) were packaged into AAV serotype DJ (AAV-CRISPR), and applied the viruses to differentiated C2C12 myotubes, FIG. 18. AAV-CRISPR transduced the multinucleated myotubes, and modified all endogenous genes tested. See FIG. 19, FIG. 20A, and FIG. 20B. Mutation frequencies increased with viral dose, but began to plateau at higher doses (˜6% of Mstn edited), suggesting dose-saturation or recalcitrant cellular sub-populations. AAV-CRISPR likewise targeted tail-tip fibroblasts and GC-1 spermatogonial cells (24.6% of Mstn edited). See FIG. 20B and FIG. 21. Hence, AAV-CRISPR is functionally robust in three distinct cell types of proliferative and terminally differentiated states.

### Cell Line #1: C2C12 Myotubes

For differentiation of C2C12 into myotubes, 2×10⁴ cells were plated per well in a 96-well plate, in 100 μl of growth media. At confluency 1-2 day(s) after plating, media was replaced with fresh differentiation media (DMEM+glutaMAX+2% donor horse serum), and further incubated for 4 days. Fresh differentiation media was replaced before transduction. For transduction with AAV-containing lysates, 50 μl of each lysate was added per well. For transduction with purified AAV, the stated titers of vector genomes (vg) were added per well. Culture media was replaced with fresh differentiation media the next day, and cells were incubated for stated durations. See FIG. 19, FIG. 20A, and FIG. 20B.

### Cell Line #2: Ai9 Reporter Fibroblasts (Aka 3×Stop-tdTomato Cell Line)

The 3×Stop-tdTomato reporter cell line was derived from tail-tip fibroblasts of Ai9 mouse (JAX 007905), and immortalized with lentiviruses encoding the large SV40 T-antigen (GenTarget Inc, LVP016-Puro). Cells were cultured in DMEM+pyruvate+glutaMAX+10% FBS. For transduction with AAVs, cells were plated at 2×10⁴ per well in a 96-well plate, in 100 μl of growth media. Iodixanol-purified AAVs were applied at confluency of 70-90%. Culture media was replaced with fresh growth media the next day. Images were taken 7 days post-transduction. Transduction of Ai9 tail-tip fibroblasts with 1E12 (total vg) of AAV-CRISPR targeting the 3×Stop cassette induced excision-dependent fluorescence activation (n=2 transductions). gRNA pairs and Cas9N:Cas9C-P2A-turboGFP ratios are indicated. Td5 and TdL target 5′ of 3×Stop; Td3 and TdR target 3′ of 3×Stop. TdTomato was not observed in negative controls transduced with 6.7E11 (total vg) of Cas9C-P2A-turboGFP only. See FIG. 22.

### Cell Line #3: GC-1 Spermatogonial Stem Cells

The GC-1 spg mouse spermatogonial cell line (CRL-2053) was obtained from the American Tissue Collection Center (ATCC, Manassas, Va.), and cultured in DMEM+pyruvate+glutaMAX+10% FBS. Stated total volumes of AAV-CRISPR-containing lysates were applied to the cells 1 day post-plating. Fresh media was replaced the next day. AAV-CRISPR^{M3+M4} edits the Mstn gene in GC-1 spermatogonial cells (Cas9N:Cas9C, 1:1) (*, P<0.05, Welch's t-test, Bonferroni corrected). See FIG. 21.

### Example XIV

### Split-Cas9 Transduced as AAVs in Whole Animals—Viral Delivery Efficiency Directly Determines the Rate of Gene-Editing

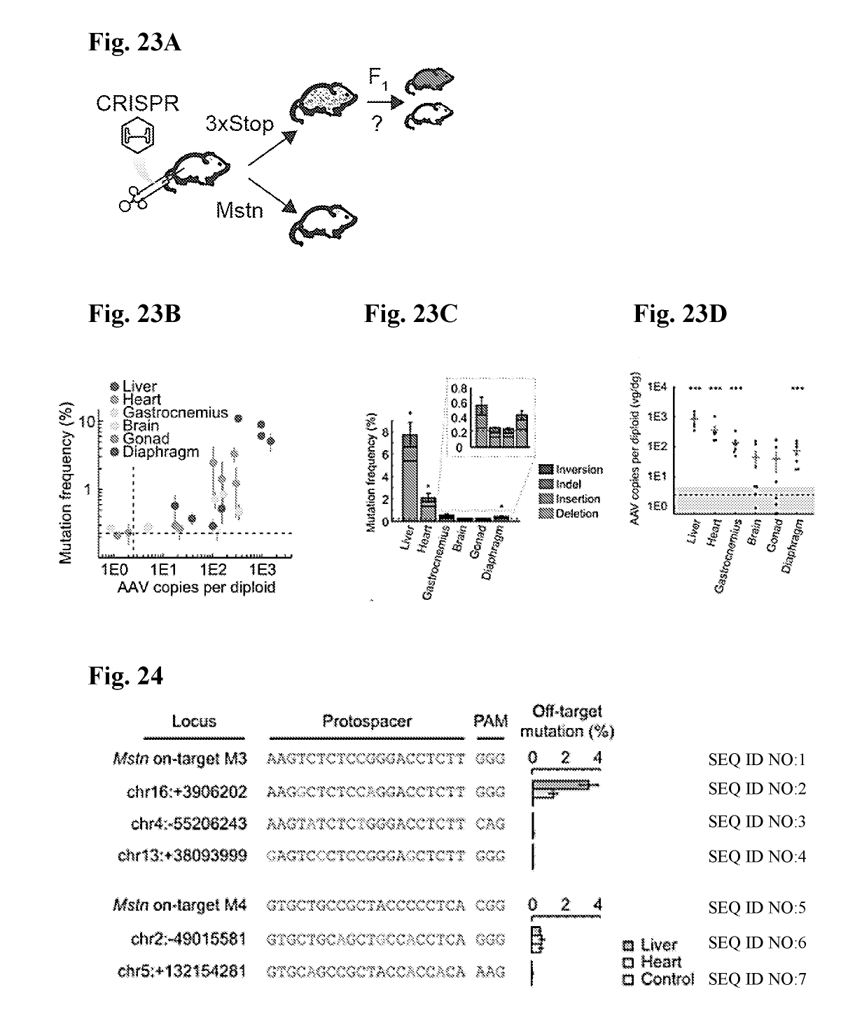

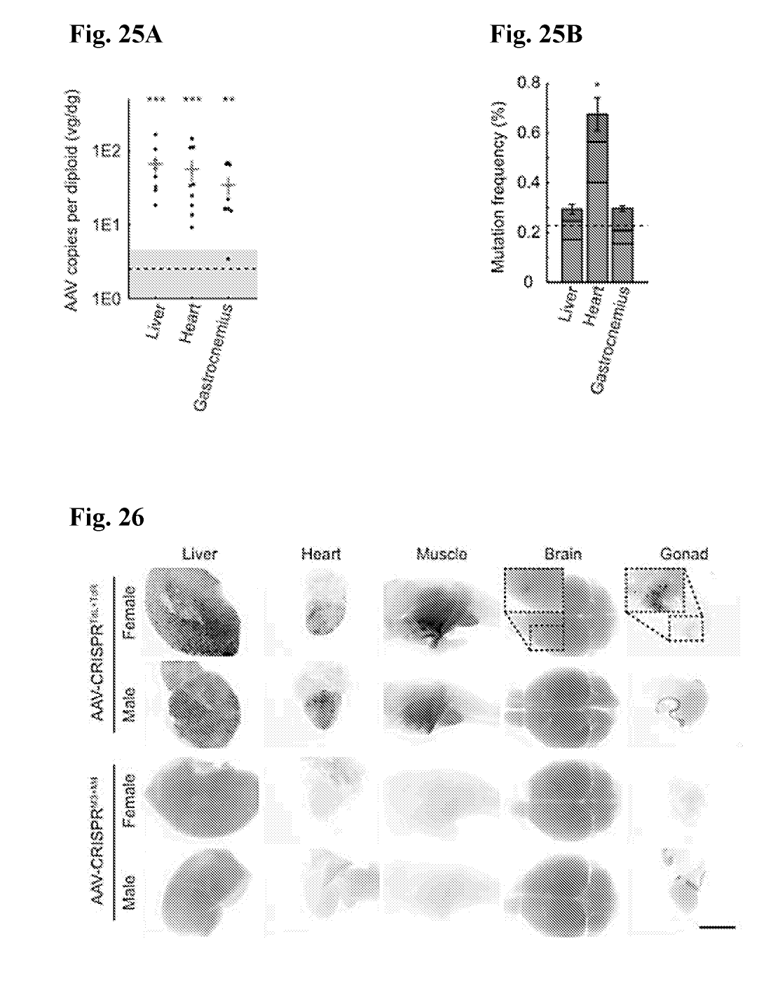

The viruses were pseudotyped to AAV serotype 9, and injected AAV9-CRISPR targeting Mstn (AAV9-CRISPR^{M3+M4}) systemically into neonatal mice, FIG. 23A. All AAV experiments were conducted in a randomized and double-blind fashion. Deep-sequencing of whole tissues from injected mice revealed genomic modifications in the liver (7.8%) and heart (2.1%), FIG. 23C, with editing frequencies in the gastrocnemius muscle (0.57%), diaphragm (0.44%), brain (0.27%), and gonads (0.25%) near the sensitivity limits of sequencing [0.23+0.07 (s.d.) %]. The only bonafide genomic off-target site identified exhibited a similar inter-tissue targeting bias. See FIG. 24. Differences in gene-targeting potentially reflect differential susceptibility of tissues to AAV transduction. To test this hypothesis, AAV vector genome (vg) copies were quantified against the mouse diploid genome (dg) by quantitative PCR. Consistent with previous studies, see Zincarelli, C., et al., Analysis of AAV serotypes 1-9 mediated gene expression and tropism in mice after systemic injection. Mol Ther, 2008. 16(6): p. 1073-80 and Inagaki, K., et al., Robust systemic transduction with AAV9 vectors in mice: efficient global cardiac gene transfer superior to that of AAV8. Mol Ther, 2006. 14(1): p. 45-53., AAV9 exhibits preferential tropism for liver, heart, and skeletal muscle (vg/dg of 850, 370, 140, respectively), while low vg copy numbers were also detected in the brain and gonads, FIG. 23D. Strikingly, gene-editing frequencies correlated strongly with AAV vector copies (r=0.73, p=0.74, P<0.05), FIG. 23B, indicating that delivery efficiency dictates mutation rate. Correspondingly, titrating the injected AAV dose modulates transduction and editing frequencies, FIG. 25A and FIG. 25B, across organs.

The CRISPR activity within each tissue can be predicted based on how well the AAV serotype transduces the said tissue(s). Many AAV serotypes exist, and they exhibit different infection profiles. Production of AAVs of desired serotype is easily done by using pRepCap expressing the desired capsid during AAV production. Hence, pseudotyping of the AAV-CRISPR to a serotype that efficiently transduces the organ/tissue of interest is critical for editing success. Conversely, to limit tissue-level off-targeting, one would use serotypes that do not transduce inappropriate tissue types.

Ai9 mice (JAX No. 007905) were used for all AAV experiments. All AAV9-CRISPR injections utilized a Cas9^{N}-gRNAs:Cas9^{C}-P2A-turboGFP ratio of 1:1. 3-day old neonates were each intraperitoneally injected with 4E12 or 5E11 vector genomes (vg) of total AAV9. Vector volumes were kept at 100 μl. Animals were euthanized via CO₂ asphyxia and cervical dislocation 3 weeks following injections. For deep sequencing of whole tissues, samples were taken from the heart body wall, liver, gastrocnemius muscle, olfactory bulb, ovary, testis, and diaphragm.

Bulk tissues were each placed in 100 μl of QuickExtract DNA Extraction Solution, and heated at 65° C. for 15 min., 95° C. for 10 min. 0.5 μl of lysate was used per 25 μl PCR reaction, and thermocycled for 25 cycles.

### Locus-Specific Amplification Primers for Deep Sequencing:

| Target | |
|---|---|
| locus | Sequence |
| Mstn F | CTTTCCCTACACGACGCTCTTCCGATCTNNNNNNGGGCCA |
| | TGAAAGGAAAAATGAAGT (SEQ ID NO: 56) |
| Mstn R | GGAGTTCAGACGTGTGCTCTTCCGATCTGCCTCTGGGGTT |
| | TGCTTGGT (SEQ ID NO: 57) |

For barcoding for deep sequencing, 1 μl of each unpurified PCR reaction was added to 20 μl of barcoding PCR reaction, and thermocycled [95° C. for 3 min., and 10 cycles of (95° C. for 10 s, 72° C. for 65 s)]. Amplicons were pooled, and the whole sequencing library was purified with self-made SPRI beads (9% PEG final concentration), and sequenced on a Miseq (Illumina) for 2×251 cycles. FASTQ were analyzed with BLAT (with parameters -t=dna -q=dna -tileSize=1 -stepSize=5 -oneOff=1 -repMatch=10000000 -minMatch=4 -minIdentity=90 -maxGap=3 -noHead) and post-alignment analyses performed with custom MATLAB (MathWorks) scripts. Alignments due to primer dimers were excluded by filtering off sequence alignments that did not extend >2 bp into the loci from the locus-specific primers. To minimize the impact of sequencing errors, conservative variant calling was performed by ignoring base substitutions, and calling only variants that overlap with a ±30 bp window from the designated CRISPR cut sites. Vehicle-injected controls were equally analyzed.

Each qPCR reaction consists of 1× FastStart Essential DNA Probes Master (Roche #06402682001), 100 nM of each hydrolysis probe (against the AAV ITR and the mouse Acvr2b locus), 340 nM of AAV ITR reverse primer, 100 nM each for all other forward and reverse primers, and 2.5 μl of input tissue lysate. For each qPCR run, a mastermix was first constituted before splitting 22.5 μl into each well, after which tissue lysates were added. The thermocycling conditions were: [95° C. 15 min.; 40 cycles of (95° C. 1 min., 60° C. 1 min.)]. FAM and HEX fluorescence were taken every cycle. AAV genomic copies per mouse diploid genome were calculated against standard curves.

qPCR Probes and Primers:

| Target | |
|---|---|
| locus | Sequence |
| Acw2b F | GCCTACTCGCTGCTGCCCATT |
| | (SEQ ID NO: 58) |
| Acw2b R | CCTGGAGACCCCCAAAAGCTC |
| | (SEQ ID NO: 59) |
| Acw2b probe | /5HEX/AGATCT + TC + CC + AC + |
| | TT + CA + GGT/3IABkFQ/ |
| | (SEQ ID NO: 60) |
| AAV ITR F | GGAACCCCTAGTGATGGAGTT |
| | (SEQ ID NO: 61) |
| AAV ITR R | CGGCCTCAGTGAGCGA |
| | (SEQ ID NO: 62) |
| AAV ITR probe | /56-FAM/CACTCCCTCTCTGCGCGCTCG/3BH |
| | (SEQ ID NO: 63) |

For each tissue sample, two repeated samplings were performed for qPCR and deep-sequencing, all on separate days, and the means plotted with s.e.m. Sequencing error and qPCR false positive rate were calculated similarly from two vehicle-injected negative control mice. See FIG. 23B, FIG. 23C, and FIG. 23D.

Off-target sites for Mstn gRNAs were predicted using the online CRISPR Design Tool, Hsu, P. D. et al. DNA targeting specificity of RNA-guided Cas9 nucleases. Nature biotechnology, 2013. 31: p. 827-832, (world wide website crispr.mit.edu). Off-target sites were ranked by the number of mismatches to the on-target sequence, and deep sequencing was performed on the top hits. Sequencing reads were analyzed equally between experimental samples (AAV9-CRISPR^{M3+M4}) and control samples (AAV9-CRISPR^{TdL+TdR}) using BLAT. Variant calls were performed for insertions and deletions that lie within a ±15 bp window from the potential off-target cut sites. The CRISPR off-target mutation frequency follows a similar inter-tissue bias. Chr16:+3906202 is the only detected bonafide off-target, FIG. 24. Reducing injected viral dosage 15 to 5E11 decreases transduction rate (measured by vector genomes per diploid genome), and correspondingly decreases gene-editing frequency. See FIG. 25A and FIG. 25B.

### Example XV

### Split-Cas9 Transduced as AAVs in Whole Animals—Usage of the Ai9 Reporter Mouse for Detection of CRISPR-Mediated Excision, at Single-Cell Resolution, within Whole Animals

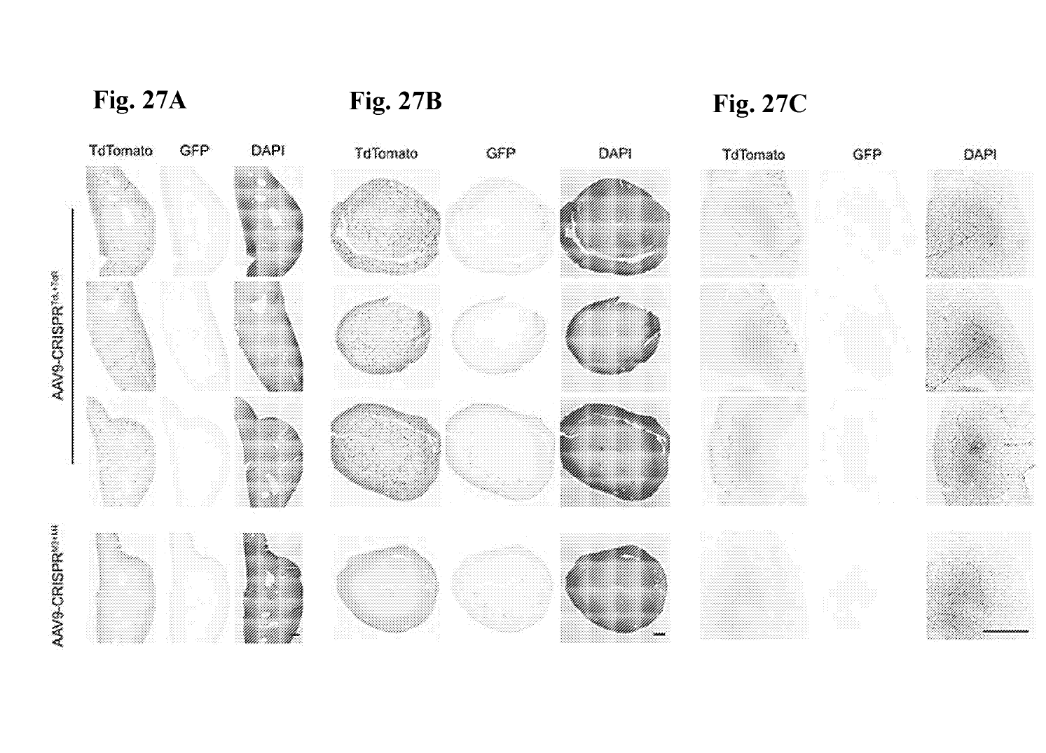

Because AAV9-CRISPR is delivered pervasively in the body, the biodistribution of CRISPR activity was examined in further detail. This is motivated in part by safety considerations for the impending human trials, where there is an urgent need to validate tissue-specificity of genetic edits by demarcating unintentionally targeted cells. Rare gene-edited cells can go undetected with deep-sequencing of bulk samples (sensitivity limits of ˜0.2%), an inadequacy in tissues where a few mutated cells could nonetheless have profound functional consequences, such as neurons, germ cells, or proto-oncogenic cells. Therefore, to determine if the sparsely transduced organs harbour mutant cells, CRISPR activity at single-cell resolution was tracked. Low (5E11) or high (4E12) doses of AAV9-CRISPR was injected targeting the 3×Stop cassette (AAV9-CRISPR^{TdL+TdR}) into neonatal Ai9 mice, FIG. 23A. Prescribed doses can range from 1E9 to 1E16, depending on the animal size. Systemic delivery of AAV9-CRISPR^{TdL+TdR} generated excision-dependent tdTomato+cells in multiple organs, which were directly observable with whole-mount microscopy, FIG. 26. In agreement with our deep-sequencing results and established AAV9 infection profile, targeted cells were predominantly found in the liver, heart, and skeletal muscle, FIG. 27A, FIG. 27B, and FIG. 27C. Notably, gene-edited cells were also detected infrequently within the brain and gonad (<0.001% of cells) (FIG. 26), at a rate that evades detection by conventional sequencing approaches. Thus, the reporter system demarcates CRISPR activity in situ with single-cell precision, and provides a rigorous tool for further evaluation of possible tissue off-targeting, such as that of unintended neuronal perturbations or germline modifications.

The reporter allows functional analyses in situ, where genetically modified cells can be examined within the native tissue context. Furthermore, the reporter provides a tool for rapid and unbiased assessment of tissue-level off-targeting, where tissue-specificity of gene-targeting can be examined throughout the whole animal. The latter is especially useful when developing therapeutics meant for subsets of diseased organ(s), for validating that AAV-CRISPR is not inadvertently acting on the other organs within the subject.

Ai9 mice (JAX No. 007905) were used for all AAV experiments. All AAV9-CRISPR injections utilized a Cas9N-gRNAs:Cas9C-P2A-turboGFP ratio of 1:1. 3-day old neonates were each intraperitoneally injected with 4E12 or 5E11 vector genomes (vg) of total AAV9. Vector volumes were kept at 100 μl. Animals were euthanized via CO2 asphyxia and cervical dislocation 3 weeks following injections. Whole organ images were taken under a fluorescent stereomicroscope.

For histology, mouse organs and tissue samples were dissected, and fixed in 4% paraformaldehyde in 1×DPBS for 1.5 hr, followed by 3 washes with 1×DPBS for 5 min. each. Samples were then immersed in 30% sucrose until submersion, embedded in O.C.T. compound (Tissue-Tek), frozen in liquid-nitrogen-cold isopantane, and cryosectioned on a Microm HM550 (Thermo Scientific). Skeletal muscles were sectioned to a thickness of 12 μm, while the liver and heart were sectioned at 20 μm. Slides were then mounted with mounting media containing DAPI (Vector Laboratories, H1500). Confocal images were taken using a Zeiss LSM780 inverted microscope. See FIG. 26, FIG. 27A, FIG. 27B, and FIG. 27C.

### Example XVI

### Maternal Transmission of AAV (Serotype 9) Harbouring Gene-Editing Tools

Gene-editing tools delivered through AAV9 are delivered to the fetus when pregnant animals were injected intravenously. This is exemplified by the detection of mosaic tdTomato+cells within the progeny when the pregnant Ai9 mothers were injected with AAV9-GFP-Cre (Cre being the gene-targeting recombinase that excises the 3×Stop cassette, activating tdTomato)

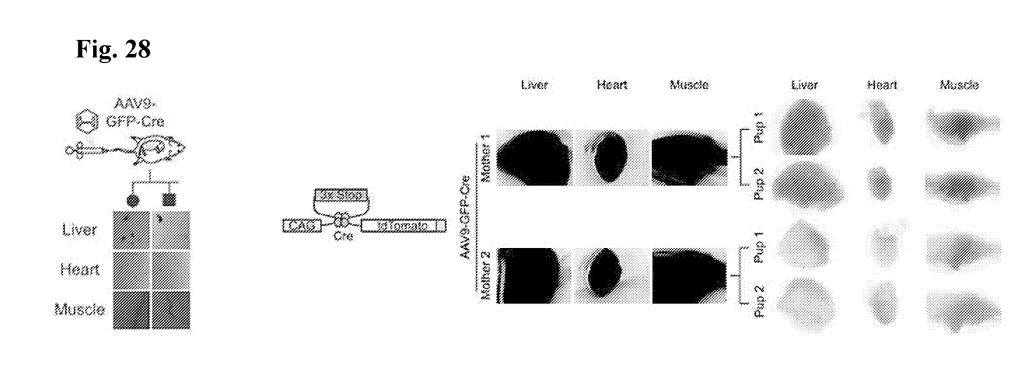

AAV9 penetrates endothelial barriers, including the blood-placental barrier, see Picconi, J. L. et al. Kidney-specific expression of GFP by in-utero delivery of pseudotyped adeno-associated virus 9. Molecular Therapy—Methods & Clinical Development, 2014. 1: p. 14014 and Okada, H., et al., Robust Long-term Transduction of Common Marmoset Neuromuscular Tissue With rAAV1 and rAAV9. Mol Ther Nucleic Acids, 2013. 2: p. e95, suggesting that gene-editing cargoes could be transmitted from mother to fetus. To assess vertical transmission of gene-editing viruses, pregnant mice (E16.5) were intravenously injected with AAV9-CRISPR^{TdL+TdR}, but did not detect tdTomato+cells within the offspring. In contrast, injecting the molar equivalent of AAV9-GFP-Cre into pregnant mice resulted in systemic loxP recombination within the mother, and mosaic genetic modifications in all progeny, FIG. 28. This indicates that viruses encoding gene-editing machinery are maternally transmissible. Therefore, with the rapidly improving efficacy of CRISPR, the risk of transmitted gene-editing must be taken into consideration.

AAV9 viruses are known to penetrate the blood-placental barrier, as detected by reporter transgenes such as GFP or lacZ. Here, gene-editing tools were examined to determine if they are active within the progeny when AAV9s are injected intravenously into the pregnant mothers. The observation of mosaic tdTomato+cells within the offspring indicates that AAV9 delivers active gene-editing tools (Cre) into the fetus. This also strongly suggests that CRISPR is likewise delivered. Gene-editing, however, has not been seen within the progeny with maternally injected AAV9-CRISPR—this is almost certainly due to CRISPR being less efficient than Cre at current efficiencies. It is likely that maternal transmission will be observed post-AAV9-CRISPR injection when AAV9-CRISPR dosage is increased, or when intrinsic efficiency of CRISPR increases with further optimization.

Pregnant dams were identified by visual inspection for vaginal plugs (E0.5). Pregnant female mice were injected via the tail vein at E16.5. 4E12 (vg) of AAV9-CRISPR or 2E12 (vg) of AAV9-GFP-Cre were injected. AAV2/9-CMV-hGHintron-GFP-Cre-WPRE-SV40pA (Lot V4565MI-R, 3.91E13 GC/ml) was obtained from the University of Pennsylvania Vector Core. The injected volume was kept at 150 μl. Mothers and pups were euthanized at 1 month after birth. Whole-organ images were taken under a fluorescent stereomicroscope. See FIG. 28.

### Example XVII

### Increased Intramuscular Expression of Cas9 Delivered as AAV-Split Cas9 Versus Electroporated Plasmid-Cas9FL

To determine expression levels of Cas9 in vivo, protein lysates from bulk muscle tissues were quantified through Western blots. Each tibialis anterior muscle of 11-week old C57BL/6 mice was injected with 4E12 of AAV9-split-Cas9, intramuscularly electroporated with 30 μg of plasmid-Cas9FL, or mock injected/electroporated with vehicle. Muscles were harvested 2 weeks post-injection, ˜10 mm³ tissue clippings were flash-frozen in liquid nitrogen, followed by lysis in 300-500 μl of T-PER Tissue Protein Extraction Solution (Thermo Scientific) supplemented with 1× Complete Protease Inhibitor (Roche), and homogenized in gentleMACS M tubes (Miltenyi Biotec). 10-15 μl of each tissue lysate was ran on 8% Bolt Bis-Tris Plus gels (Life Technologies) in 1× Bolt MOPS SDS running buffer at 165 V for 50 mins. Protein transfer was performed with iBlot (Life Technologies) onto PVDF membranes, using program 3 for 13 mins. Western blots were conducted with 1:200 of anti-Cas9 polyclonal antibody (Clontech 632607), 1:400 of anti-GAPDH polyclonal antibody (Santa Cruz sc-25778), and 1:2500 of anti-rabbit IgG-HRP secondary antibody (Santa Cruz sc-2004), using an iBind device (Life Technologies). Stained membranes were developed with SuperSignal West Femto Maximum Sensitivity Subtrate (Thermo Scientific) and imaged on Chemidoc MP (Bio-Rad). Band intensities were quantified with ImageJ software.

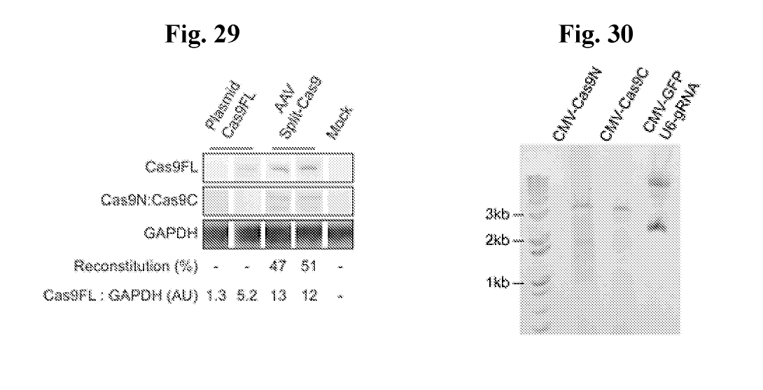

As shown in FIG. 29, split-Cas9 reconstitutes Cas9FL at 50% efficiency when delivered intramuscularly. AAV9-split-Cas9 expresses reconstituted Cas9FL at levels >2-fold higher than that from electroporated plasmid-Cas9FL.

### Example XVIII

### Split Cas9 can be Packaged into Self-Complementary AAV (scAAV)

Split-Cas9 can be packaged into self-complementary AAV (scAAV), a robust delivery platform that expresses the payload stronger and faster. Correspondingly, the higher transduction efficiency would enable administration of lower dosages (5-100 fold lower), while achieving similar expression levels as single-stranded AAVs (ssAAV). Useful scAAV are disclosed in McCarty, D. M., P. E. Monahan, and R. J. Samulski. “Self-complementary recombinant adeno-associated virus (scAAV) vectors promote efficient transduction independently of DNA synthesis.” Gene therapy 8.16 (2001): 1248-1254; McCarty, D. M., et al. “Adeno-associated virus terminal repeat (TR) mutant generates self-complementary vectors to overcome the rate-limiting step to transduction in vivo.” Gene therapy 10.26 (2003): 2112-2118. Gao, Guang-Ping, et al. “High-level transgene expression in nonhuman primate liver with novel adeno-associated virus serotypes containing self-complementary genomes.” Journal of virology 80.12 (2006): 6192-6194; Wang, Z., et al. “Rapid and highly efficient transduction by double-stranded adeno-associated virus vectors in vitro and in vivo.” Gene therapy 10.26 (2003): 2105-2111 and Jianqing, et al. “Self-complementary recombinant adeno-associated viral vectors: packaging capacity and the role of rep proteins in vector purity.” Human gene therapy 18.2 (2007): 171-182 each of which are hereby incorporated by reference in their entireties. The key feature of scAAV is the mutation in one of its ITRs, which generates dsDNA viral genomes instead of the normal ssDNA genomes. This means the payload exists in a transcriptionally active state, without the need for double-strand conversion within the host that is a major rate-limiting step of AAV gene delivery. However, because AAV viruses are spatially compact, the packaging of dsDNA would reduce the payload capacity by half (2.2-2.4 kb). A report (Wu, 2007) has claimed that the upper limit is higher than expected, at 3.3 kb, but importantly, because all known Cas9 orthologs are larger than 2.95 kb, this means none can be trivially packaged as a full-length transgene. Split-Cas9 reduces the S. pyogenes Cas9 coding sequences to 2.5 kb and 2.2 kb respectively, smaller than that of all known orthologs. Splitting or truncating the other Cas9 orthologs would be necessary for this route of administration.

Each half of the split-Cas9 was cloned into a scAAV plasmid vector. scAAV viruses were produced via the triple-transfection method, and purified by density gradient ultracentrifugation. To determine the integrity of the viral genomes, purified viruses were lysed with proteinase K, and gel electrophoresis was performed with the lysates.

As shown in FIG. 30, scAAV can accommodate payloads larger than the 2.2 kb-2.4 kb as commonly stated. The presence of viral genomic bands at the expected 3.2 kb and 2.8 kb sizes for scAAV-Cas9N and scAAV-Cas9C indicates that the split-Cas9 components were packaged into the viruses intact. Sequences are provided below.

scAA V-Promoter-Cas9N-RmaIntN-synpA (3.2 kb):

CAGCAGCTGCGCGCTCGCTCGCTCACTGAGGCCGCCCGGGCAAAGCCCGGGCGTCGGGC GACCTTTGGTCGCCCGGCCTCAGTGAGCGAGCGAGCGCGCAGAGAGGGAGTGGGGTTAA ACGTTGACATTGATTATTGACTAGCCGCTAGCAGGACTCACGGGGATTTCCAAGTCTC CACCCCATTGACGTCAATGGGAGTTTGTTTTGGCACCAAAATCAACGGGACTTTCCAA AATGTCGTAACAACTCCGCCCCATTGACGCAAATGGGCGGTAGGCGTGTACGGTGGG AGGTCTATATAAGCAGAGCTCGTTTAGTGAACCGTCAGATCGCCTGGAGACGCCATCC GGACTCTAAGGTAAATATAAAATTTTTAAGTGTATAATGTGTTAAACTACTGATTCTA ATTGTTTCTCTCTTTTAGATTCCAACCTTTGGAACTGAATTCGCCGCCACCATGGCCCC AAAGAAGAAGCGGAAGGTCGGTATCCACGGAGTCCCAGCAGCCGACAAGAAGTACTC CATTGGGCTCGATATCGGCACAAACAGCGTCGGCTGGGCCGTCATTACGGACGAGTAC AAGGTGCCGAGCAAAAAATTCAAAGTTCTGGGCAATACCGATCGCCACAGCATAAAG AAGAACCTCATTGGCGCCCTCCTGTTCGACTCCGGGGAAACGGCCGAAGCCACGCGGC TCAAAAGAACAGCACGGCGCAGATATACCCGCAGAAAGAATCGGATCTGCTACCTGC AGGAGATCTTTAGTAATGAGATGGCTAAGGTGGATGACTCTTTCTTCCATAGGCTGGA GGAGTCCTTTTTGGTGGAGGAGGATAAAAAGCACGAGCGCCACCCAATCTTTGGCAAT ATCGTGGACGAGGTGGCGTACCATGAAAAGTACCCAACCATATATCATCTGAGGAAG AAGCTTGTAGACAGTACTGATAAGGCTGACTTGCGGTTGATCTATCTCGCGCTGGCGC ATATGATCAAATTTCGGGGACACTTCCTCATCGAGGGGGACCTGAACCCAGACAACAG CGATGTCGACAAACTCTTTATCCAACTGGTTCAGACTTACAATCAGCTTTTCGAAGAG AACCCGATCAACGCATCCGGAGTTGACGCCAAAGCAATCCTGAGCGCTAGGCTGTCCA AATCCCGGCGGCTCGAAAACCTCATCGCACAGCTCCCTGGGGAGAAGAAGAACGGCC TGTTTGGTAATCTTATCGCCCTGTCACTCGGGCTGACCCCCAACTTTAAATCTAACTTC GACCTGGCCGAAGATGCCAAGCTTCAACTGAGCAAAGACACCTACGATGATGATCTC GACAATCTGCTGGCCCAGATCGGCGACCAGTACGCAGACCTTTTTTTGGCGGCAAAGA ACCTGTCAGACGCCATTCTGCTGAGTGATATTCTGCGAGTGAACACGGAGATCACCAA AGCTCCGCTGAGCGCTAGTATGATCAAGCGCTATGATGAGCACCACCAAGACTTGACT TTGCTGAAGGCCCTTGTCAGACAGCAACTGCCTGAGAAGTACAAGGAAATTTTCTTCG ATCAGTCTAAAAATGGCTACGCCGGATACATTGACGGCGGAGCAAGCCAGGAGGAAT TTTACAAATTTATTAAGCCCATCTTGGAAAAAATGGACGGCACCGAGGAGCTGCTGGT AAAGCTTAACAGAGAAGATCTGTTGCGCAAACAGCGCACTTTCGACAATGGAAGCAT CCCCCACCAGATTCACCTGGGCGAACTGCACGCTATCCTCAGGCGGCAAGAGGATTTC TACCCCTTTTTGAAAGATAACAGGGAAAAGATTGAGAAAATCCTCACATTTCGGATAC CCTACTATGTAGGCCCCCTCGCCCGGGGAAATTCCAGATTCGCGTGGATGACTCGCAA ATCAGAAGAGACCATCACTCCCTGGAACTTCGAGGAAGTCGTGGATAAGGGGGCCTC TGCCCAGTCCTTCATCGAAAGGATGACTAACTTTGATAAAAATCTGCCTAACGAAAAG GTGCTTCCTAAACACTCTCTGCTGTACGAGTACTTCACAGTTTATAACGAGCTCACCAA GGTCAAATACGTCACAGAAGGGATGAGAAAGCCAGCATTCCTGTCTGGAGAGCAGAA GAAAGCTATCGTGGACCTCCTCTTCAAGACGAACCGGAAAGTTACCGTGAAACAGCTC AAAGAAGACTATTTCAAAAAGATTGAATGTTTCGACTCTGTTGAAATCAGCGGAGTGG AGGATCGCTTCAACGCATCCCTGGGAACGTATCACGATCTCCTGAAAATCATTAAAGA CAAGGACTTCCTGGACAATGAGGAGAACGAGGACATTCTTGAGGACATTGTCCTCACC CTTACGTTGTTTGAAGATAGGGAGATGATTGAAGAACGCTTGAAAACTTACGCTCATC TCTTCGACGACAAAGTCATGAAACAGCTCAAGAGGCGCCGATATACAGGATGGGGGC GGCTGTCAAGAAAACTGATCAATGGGATCCGAGACAAGCAGAGTGGAAAGACAATCC TGGATTTTCTTAAGTCCGATGGATTTGCCAACCGGAACTTCATGCAGTTGATCCATGAT GACTCTCTCACCTTTAAGGAGGACATCCAGAAAGCACAAGTTTGTCTGGCTGGCGATA CTCTCATTACCCTGGCCGATGGACGACGAGTGCCTATTAGAGAACTGGTGTCACAGCA GAATTTTTCCGTGTGGGCTCTGAATCCTCAGACTTACCGCCTGGAGAGGGCTAGAGTG AGTAGAGCTTTCTGTACCGGCATCAAACCTGTGTACCGCCTCACCACTAGACTGGGGA GATCCATTAGGGCCACTGCCAACCACCGATTTCTCACACCTCAGGGCTGGAAACGAGT CGATGAACTCCAGCCTGGAGATTACCTGGCTCTGCCTAGGAGAATCCCTACTGCCTCC TGACAATAAAATATCTTATTTTCATTACATCTGTGTGTTGGTTTTTTGTGTAGCTAGCGCGT AGATAAGTAGCATGGCGGGTTAATCATTAACTACAAGGAACCCCTAGTGATGGAGTTGG CCACTCCCTCTCTGCGCGCTCGCTCGCTCACTGAGGCCGGGCGACCAAAGGTCGCCCGAC GCCCGGGCTTTGCCCGGGCGGCCTCAGTGAGCGAGCGAGCGCGCCAG (SEQ ID NO:64)

scAA V-Promoter-RmaIntC-Cas9C-synpA (2.8 kb):

CAGCAGCTGCGCGCTCGCTCGCTCACTGAGGCCGCCCGGGCAAAGCCCGGGCGTCGGGC GACCTTTGGTCGCCCGGCCTCAGTGAGCGAGCGAGCGCGCAGAGAGGGAGTGGGGTTAA ACGTTGACATTGATTATTGACTAGCCGCTAGCAGGACTCACGGGGATTTCCAAGTCTC CACCCCATTGACGTCAATGGGAGTTTGTTTTGGCACCAAAATCAACGGGACTTTCCAA AATGTCGTAACAACTCCGCCCCATTGACGCAAATGGGCGGTAGGCGTGTACGGTGGG AGGTCTATATAAGCAGAGCTCGTTTAGTGAACCGTCAGATCGCCTGGAGACGCCATCC GGACTCTAAGGTAAATATAAAATTTTTAAGTGTATAATGTGTTAAACTACTGATTCTA ATTGTTTCTCTCTTTTAGATTCCAACCTTTGGAACTGAATTCGCCGCCACCATGGCGGC GGCGTGCCCGGAACTGCGTCAGCTGGCGCAGAGCGATGTGTATTGGGATCCGATTGTG AGCATTGAACCGGATGGCGTGGAAGAAGTGTTTGATCTGACCGTGCCGGGCCCGCATA ACTTTGTGGCGAACGATATTATTGCGCATAACTCTGGCCAGGGGGACAGTCTTCACGA GCACATCGCTAATCTTGCAGGTAGCCCAGCTATCAAAAAGGGAATACTGCAGACCGTT AAGGTCGTGGATGAACTCGTCAAAGTAATGGGAAGGCATAAGCCCGAGAATATCGTT ATCGAGATGGCCCGAGAGAACCAAACTACCCAGAAGGGACAGAAGAACAGTAGGGA AAGGATGAAGAGGATTGAAGAGGGTATAAAAGAACTGGGGTCCCAAATCCTTAAGGA ACACCCAGTTGAAAACACCCAGCTTCAGAATGAGAAGCTCTACCTGTACTACCTGCAG AACGGCAGGGACATGTACGTGGATCAGGAACTGGACATCAATCGGCTCTCCGACTAC GACGTGGATCATATCGTGCCCCAGTCTTTTCTCAAAGATGATTCTATTGATAATAAAGT GTTGACAAGATCCGATAAAAATAGAGGGAAGAGTGATAACGTCCCCTCAGAAGAAGT TGTCAAGAAAATGAAAAATTATTGGCGGCAGCTGCTGAACGCCAAACTGATCACACA ACGGAAGTTCGATAATCTGACTAAGGCTGAACGAGGTGGCCTGTCTGAGTTGGATAAA GCCGGCTTCATCAAAAGGCAGCTTGTTGAGACACGCCAGATCACCAAGCACGTGGCCC AAATTCTCGATTCACGCATGAACACCAAGTACGATGAAAATGACAAACTGATTCGAG AGGTGAAAGTTATTACTCTGAAGTCTAAGCTGGTCTCAGATTTCAGAAAGGACTTTCA GTTTTATAAGGTGAGAGAGATCAACAATTACCACCATGCGCATGATGCCTACCTGAAT GCAGTGGTAGGCACTGCACTTATCAAAAAATATCCCAAGCTTGAATCTGAATTTGTTT ACGGAGACTATAAAGTGTACGATGTTAGGAAATGATCGCAAAGTCTGAGCAGGAAA TAGGCAAGGCCACCGCTAAGTACTTCTTTTACAGCAATATTATGAATTTTTTCAAGACC GAGATTACACTGGCCAATGGAGAGATTCGGAAGCGACCACTTATCGAAACAAACGGA GAAACAGGAGAAATCGTGTGGGACAAGGGTAGGGATTTCGCGACAGTCCGGAAGGTC CTGTCCATGCCGCAGGTGAACATCGTTAAAAAGACCGAAGTACAGACCGGAGGCTTCT CCAAGGAAAGTATCCTCCCGAAAAGGAACAGCGACAAGCTGATCGCACGCAAAAAAG ATTGGGACCCCAAGAAATACGGCGGATTCGATTCTCCTACAGTCGCTTACAGTGTACT GGTTGTGGCCAAAGTGGAGAAAGGGAAGTCTAAAAAACTCAAAAGCGTCAAGGAACT GCTGGGCATCACAATCATGGAGCGATCAAGCTTCGAAAAAAACCCCATCGACTTTCTC GAGGCGAAAGGATATAAAGAGGTCAAAAAAGACCTCATCATTAAGCTTCCCAAGTAC TCTCTCTTTGAGCTTGAAAACGGCCGGAAACGAATGCTCGCTAGTGCGGGCGAGCTGC AGAAAGGTAACGAGCTGGCACTGCCCTCTAAATACGTTAATTTCTTGTATCTGGCCAG CCACTATGAAAAGCTCAAAGGGTCTCCCGAAGATAATGAGCAGAAGCAGCTGTTCGT GGAACAACACAAACACTACCTTGATGAGATCATCGAGCAAATAAGCGAATTCTCCAA AAGAGTGATCCTCGCCGACGCTAACCTCGATAAGGTGCTTTCTGCTTACAATAAGCAC AGGGATAAGCCCATCAGGGAGCAGGCAGAAAACATTATCCACTTGTTTACTCTGACCA ACTTGGGCGCGCCTGCAGCCTTCAAGTACTTCGACACCACCATAGACAGAAAGCGGTA CACCTCTACAAAGGAGGTCCTGGACGCCACACTGATTCATCAGTCAATTACGGGGCTC TATGAAACAAGAATCGACCTCTCTCAGCTCGGTGGAGACAGCAGGGCTGACCCCAAG AAGAAGAGGAAGGTGTGACAATAAAATATCTTTATTTTCATTACATCTGTGTGTTGGTTTTTT GTGTTAGCTAGCGCGTAGATAAGTAGCATGGCGGGTTAATCATTAACTACAAGGAACC CCTAGTGATGGAGTTGGCCACTCCCTCTCTGCGCGCTCGCTCGCTCACTGAGGCCGGGCG ACCAAAGGTCGCCCGACGCCCGGGCTTTGCCCGGGCGGCCTCAGTGAGCGAGCGAGCGC GCCAG (SEQ ID NO:65)

scAA V-Promoter-GFP-SV40 pA-U6-gRNA (2.0 kb):

CAGCAGCTGCGCGCTCGCTCGCTCACTGAGGCCGCCCGGGCAAAGCCCGGGCGTCGGGC GACCTTTGGTCGCCCGGCCTCAGTGAGCGAGCGAGCGCGCAGAGAGGGAGTGGGGTTAA ACGTTGACATTGATTATTGACTAGCCGCTAGCAGGACTCACGGGGATTTCCAAGTCTC CACCCCATTGACGTCAATGGGAGTTTGTTTTGGCACCAAAATCAACGGGACTTTCCAA AATGTCGTAACAACTCCGCCCCATTGACGCAAATGGGCGGTAGGCGTGTACGGTGGG AGGTCTATATAAGCAGAGCTCGTTTAGTGAACCGTCAGATCGCCTGGAGACGCCATCC GGACTCTAAGGTAAATATAAAATTTTTAAGTGTATAATGTGTTAAACTACTGATTCTA ATTGTTTCTCTCTTTTAGATTCCAACCTTTGGAACTGAATTCCGCGGGCCCGGGATCCA CCGGTCGCCACCATGGTGAGCAAGGGCGAGGAGCTGTTCACCGGGGTGGTGCCCATC CTGGTCGAGCTGGACGGCGACGTAAACGGCCACAAGTTCAGCGTGTCCGGCGAGGGC GAGGGCGATGCCACCTACGGCAAGCTGACCCTGAAGTTCATCTGCACCACCGGCAAG CTGCCCGTGCCCTGGCCCACCCTCGTGACCACCCTGACCTACGGCGTGCAGTGCTTCA GCCGCTACCCCGACCACATGAAGCAGCACGACTTCTTCAAGTCCGCCATGCCCGAAGG CTACGTCCAGGAGCGCACCATCTTCTTCAAGGACGACGGCAACTACAAGACCCGCGCC GAGGTGAAGTTCGAGGGCGACACCCTGGTGAACCGCATCGAGCTGAAGGGCATCGAC TTCAAGGAGGACGGCAACATCCTGGGGCACAAGCTGGAGTACAACTACAACAGCCAC AACGTCTATATCATGGCCGACAAGCAGAAGAACGGCATCAAGGTGAACTTCAAGATC CGCCACAACATCGAGGACGGCAGCGTGCAGCTCGCCGACCACTACCAGCAGAACACC CCCATCGGCGACGGCCCCGTGCTGCTGCCCGACAACCACTACCTGAGCACCCAGTCCG CCCTGAGCAAAGACCCCAACGAGAAGCGCGATCACATGGTCCTGCTGGAGTTCGTGA CCGCCGCCGGGATCACTCTCGGCATGGACGAGCTGTACAAGTAAAGCGGCCGCTAGG CCTCACCTGCGATCTCGATGCTTTATTTGTGAAATTTGTGATGCTATTGCTTTATTTGTAACC ATTATAAGCTGCAATAAACAAGTTAACAACAACAATTGCATTCATTITATGTTTCAGGTTCAGG GGGAGGTGTGGGAGGTTTTTTAAACTAGTTGTACAAAAAAGCAGGCTTTAAAGGAACCAATT CAGTCGACTGGATCCGGTACCAAGGTCGGGCAGGAAGAGGGCCTATTTCCCATGATTCCTT CATATTTGCATATACGATACAAGGCTGTTAGAGAGATAATTAGAATTAATTTGACTGTAAACAC AAAGATATTAGTACAAAATACGTGACGTAGAAAGTAATAATTTCTTGGGTAGTTTGCAGTTTTA AAATTATGTTTTAAAATGGACTATCATATGCTTACCGTAACTTGAAAGTATTTCGATTTCTTGG CTTTATATATCTTGTGGAAAGGACGAAACACCG[spacer]GTTTTAGAGCTAGAAATAGCAAGTT AAAATAAGGCTAGTCCGTTATCAACTTGAAAAAGTGGCACCGAGTCGGTGCTTTTTTTTAGCT AGCGCGTAGATAAGTAGCATGGCGGGTTAATCATTAACTACAAGGAACCCCTAGTGATG GAGTTGGCCACTCCCTCTCTGCGCGCTCGCTCGCTCACTGAGGCCGGGCGACCAAAGGTC GCCCGACGCCCGGGCTTTGCCCGGGCGGCCTCAGTGAGCGAGCGAGCGCGCCAG

(SEQ ID NO:66)

1. A method of providing a cell with a Cas9 protein comprising providing to the cell a first nucleic acid encoding a first portion of the Cas9 protein and a second nucleic acid encoding a second portion of the Cas9 protein,
wherein the cell expresses the first nucleic acid encoding the first portion of the Cas9 protein,
wherein the cell expresses the second nucleic acid encoding the second portion of the Cas9 protein, and
wherein the first portion of the Cas9 protein and the second portion of the Cas9 protein are joined together to form the Cas9 protein.

2. The method of claim 1 wherein the first nucleic acid and the second nucleic acid are delivered to the cell by separate vectors.

3. The method of claim 1 wherein the first nucleic acid is delivered to the cell by a plasmid or adeno-associated virus.

4. The method of claim 1 wherein the second nucleic acid is delivered to the cell by a plasmid or an adeno-associated virus.

5. The method of claim 1 wherein the Cas9 is a Type II CRISPR system Cas9.

6. The method of claim 1 wherein the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

7. The method of claim 1 wherein the first nucleic acid encodes a first portion of the Cas9 protein having a Rhodothermus marinus N-split-intein RmaIntN and wherein the second nucleic acid encodes a second portion of the Cas9 protein having a Rhodothermus marinus C-split-intein RmaIntC and wherein the first portion of the Cas9 protein and the second portion of the Cas9 protein are joined together to form the Cas9 protein.

8. The method of claim 1 wherein the first portion of the Cas9 protein is the N-terminal lobe of the Cas9 protein and the second portion of the Cas9 protein is the C-terminal lobe of the Cas9 protein.

9. The method of claim 1 wherein the first portion of the Cas9 protein is the N-terminal lobe of the Cas9 protein up to amino acid V713 and the second portion of the Cas9 protein is the C-terminal lobe of the Cas9 protein beginning at D714.

10. The method of claim 1 wherein the cell is a eukaryotic cell or prokaryotic cell.

11. The method of claim 1 wherein the cell is a bacteria cell, yeast cell, a mammalian cell, a plant cell or an animal cell.

12. The method of claim 1 wherein the Cas9 protein is an enzymatically active Cas9 protein, a Cas9 protein nickase or a nuclease null Cas9 protein.

13. A method of altering a target nucleic acid in a cell comprising
providing to the cell a first nucleic acid encoding a first portion of a Cas9 protein and a second nucleic acid encoding a second portion of the Cas9 protein,
providing to the cell a third nucleic acid encoding RNA complementary to the target nucleic acid,
wherein the cell expresses the RNA, the first portion of the Cas9 protein and the second portion of the Cas9 protein, and
wherein the first portion of the Cas9 protein and the second portion of the Cas9 protein are joined together to form the Cas9 protein,
wherein the RNA and the Cas9 protein form a co-localization complex with the target nucleic acid.

14. The method of claim 13 wherein the Cas9 protein is enzymatically active and the enzymatically active Cas9 protein cleaves the target nucleic acid in a site specific manner.

15. The method of claim 13 wherein the first nucleic acid and the second nucleic acid are delivered to the cell by separate vectors.

16. The method of claim 13 wherein the first nucleic acid is delivered to the cell by a plasmid or adeno-associated virus.

17. The method of claim 13 wherein the second nucleic acid is delivered to the cell by a plasmid or an adeno-associated virus.

18. The method of claim 13 wherein the Cas9 is a Type II CRISPR system Cas9.

19. The method of claim 13 wherein the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

20. The method of claim 13 wherein the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein.

21. The method of claim 13 wherein the first portion of the Cas9 protein is the N-terminal lobe of the Cas9 protein and the second portion of the Cas9 protein is the C-terminal lobe of the Cas9 protein.

22. The method of claim 13 wherein the first portion of the Cas9 protein is the N-terminal lobe of the Cas9 protein up to amino acid V713 and the second portion of the Cas9 protein is the C-terminal lobe of the Cas9 protein beginning at D714.

23. The method of claim 13 wherein the cell is a eukaryotic cell or a prokaryotic cell.

24. The method of claim 13 wherein the cell is a bacteria cell, a yeast cell, a mammalian cell, a plant cell or an animal cell.

25. The method of claim 13 wherein the Cas9 protein is an enzymatically active Cas9 protein, a Cas9 protein nickase or a nuclease null Cas9 protein.

26. A cell comprising
a first foreign nucleic acid encoding a first portion of a Cas9 protein and a second nucleic acid encoding a second portion of the Cas9 protein, and a third foreign nucleic acid encoding one or more RNAs complementary to DNA, wherein the DNA includes a target nucleic acid,
wherein the one or more RNAs, the Cas9 protein are members of a co-localization complex for the target nucleic acid.

27. The method of claim 26 wherein the cell is a eukaryotic cell or a prokaryotic cell.

28. The method of claim 26 wherein the cell is a bacteria cell, a yeast cell, a mammalian cell, a plant cell or an animal cell.

29. A method of delivering a Cas9 protein to cells within a subject comprising
systemically administering to the subject a first nucleic acid encoding a first portion of the Cas9 protein wherein the first nucleic acid is within a first vector and
intravenously administering to the subject a second nucleic acid encoding a second portion of the Cas9 protein wherein the second nucleic acid is within a second vector,
wherein the first vector delivers the first nucleic acid to a cell and wherein the second vector delivers the second nucleic acid to the cell,
wherein the cell expresses the first nucleic acid encoding the first portion of the Cas9 protein,
wherein the cell expresses the second nucleic acid encoding the second portion of the Cas9 protein, and
wherein the first portion of the Cas9 protein and the second portion of the Cas9 protein are joined together to form the Cas9 protein.

30. The method of claim 29 wherein the first vector is a plasmid or adeno-associated virus.

31. The method of claim 29 wherein the second vector is a plasmid or adeno-associated virus.

32. The method of claim 29 wherein the Cas9 is a Type II CRISPR system Cas9.

33. The method of claim 29 wherein the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

34. The method of claim 29 wherein the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein.

35. The method of claim 29 wherein the first portion of the Cas9 protein is the N-terminal lobe of the Cas9 protein and the second portion of the Cas9 protein is the C-terminal lobe of the Cas9 protein.

36. The method of claim 29 wherein the first portion of the Cas9 protein is the N-terminal lobe of the Cas9 protein up to amino acid V713 and the second portion of the Cas9 protein is the C-terminal lobe of the Cas9 protein beginning at D714.

37. The method of claim 29 wherein the cell is a eukaryotic cell or prokaryotic cell.

38. The method of claim 29 wherein the cell is a bacteria cell, a yeast cell, a mammalian cell, a plant cell or an animal cell.

39. The method of claim 29 wherein the Cas9 protein is an enzymatically active Cas9 protein, a Cas9 protein nickase or a nuclease null Cas9 protein.

40. A method of providing a cell with a Cas9 protein comprising providing to the cell one or more foreign nucleic acids encoding a plurality of separate portions or segments of the Cas9 protein,
wherein the cell expresses the one or more foreign nucleic acids to produce the plurality of separate portions or segments of the Cas9 protein, and
wherein the plurality of separate portions or segments of the Cas9 protein are joined together to form the Cas9 protein active to colocalize with guide RNA to a target nucleic acid.

41. The method of claim 40 wherein the one or more foreign nucleic acids are delivered to the cell by separate vectors.

42. The method of claim 40 wherein the one or more foreign nucleic acids are delivered to the cell by separate plasmids or adeno-associated viruses.

43. The method of claim 40 wherein the Cas9 is a Type II CRISPR system Cas9.

44. The method of claim 40 wherein the plurality of separate portions or segments are connected or joined together by linker pairs.

45. The method of claim 40 wherein the plurality of separate portions or segments are connected or joined together by split-intein pairs.

46. The method of claim 40 wherein the cell is a eukaryotic cell or prokaryotic cell.

47. The method of claim 40 wherein the cell is a bacteria cell, a yeast cell, a mammalian cell, a plant cell or an animal cell.

48. The method of claim 40 wherein the Cas9 protein is an enzymatically active Cas9 protein, a Cas9 protein nickase or a nuclease null Cas9 protein.

49. The method of claim 8, wherein the Cas9 protein is SpCas9, AnCas9, or SaCas9.
